(19) United States
(12) Patent Application Publication
(10) Pub. No.: US 2003/0176385 A1
(43) Pub. Date: Sep. 18, 2003

(54) Antisense modulation of protein expression

(76) Inventors: Jingfang Ju, Orange, CT (US); Chunli Huang, North Haven, CT (US); Haihong Zhong, Guilford, CT (US); Jan Fredrik Simons, New Haven, CT (US); Bruce E. Taillon, Middletown, CT (US); John S. Chant, Branford, CT (US); John A. Peyman, New Haven, CT (US); Glennda Smithson, Guilford, CT (US); Isabelle Millet, Milford, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/305,810

(22) Filed: Nov. 27, 2002

Related U.S. Application Data
(63) Continuation-in-part of application No. 09/625,634, filed on Jul. 26, 2000.
(60) Provisional application No. 60/334,148, filed on Nov. 29, 2001.
(60) Provisional application No. 60/336,572, filed on Dec. 4, 2001.
(60) Provisional application No. 60/192,838, filed on Mar. 29, 2000.
(60) Provisional application No. 60/194,256, filed on Apr. 3, 2000.
(60) Provisional application No. 60/182,637, filed on Feb. 15, 2000.

(51) Int. Cl. A61K 48/00; C07H 21/04

(52) U.S. Cl. 514/44; 435/375; 536/23.2

(57) ABSTRACT

Antisense compounds, compositions and methods are provided for modulating the expression of H-Ras, WNT-7B, acetylglucosaminyltransferase, voltage-gated K channel, IL-8, ion transport, Map3K8 and Thymidine kinase. The compositions comprise antisense compounds, particularly antisense oligonucleotides, targeted to nucleic acids encoding H-Ras, WNT-7B, acetylglucosaminyltransferase, voltage-gated K channel, IL-8, ion transport, Map3K8 and Thymidine kinase. Methods of using these compounds for modulation of H-Ras, WNT-7B, acetylglucosaminyltransferase, voltage-gated K channel, IL-8, ion transport, Map3K8 and Thymidine kinase.expression and for treatment of diseases associated with expression of H-Ras, WNT-7B, acetylglucosaminyltransferase, voltage-gated K channel, 1L-8, ion transport, Map3KS and Thymidine kinase are provided.

### RELATED APPLICATIONS

This application claims the benefit of U.S. S No. 60/334,148, filed Nov. 29, 2001 and U.S. S No. 60/336,572, filed Dec. 4, 2001 and is a Continuation-in-Part of U.S. Ser. No. 09/625,634, filed Jul. 26, 2000 which claims the benefit of U.S. S No. 60/192,838, filed Mar. 29, 2000 and U.S. S No. 60/194,256, filed Apr. 3, 2000 and a Continuation-in-Part of U.S. Ser. No. 09/957,187, filed Sep. 19, 2001 which claims the benefit of U.S. S No. 60/233,798, filed Sep. 19, 2000 and a Continuation-in-Part of U.S. Ser. No. 09/970,813, filed Oct. 4, 2001 which claims the benefit of U.S. S No. 60/182,637, filed Feb. 15, 2000, and No. 60/240,316, filed Oct. 13, 2000 and a Continuation-in-Part of U.S. Ser. No. ______ [unknown], filed Apr. 2, 2002 which claims the benefit of U.S. S No. 60/282,529, filed Apr. 9, 2001 and 60/282,537, filed Apr. 9, 2001 and a Continuation-in-Part of U.S. Ser. No. 10/114,153, filed Apr. 2, 2002 which claims the benefit of U.S. S No. 60/327,448, filed Oct. 5, 2001 and a Continuation-in-Part of U.S. Ser. No. 10/136,826, filed May 1, 2002 which claims the benefit of U.S. S No. 60/288,063, filed May 2, 2001 and a Continuation-in-Part of U.S. Ser. No. ______ [unknown], filed May 1, 2002 which claims the benefit of U.S. S No. 60/327,455, filed Oct. 5, 2001 the contents of all of these applications which are incorporated herein by reference in their entireties

### FIELD OF THE INVENTION

The present invention provides compositions and methods for modulating the expression of H-Ras, WNT-7B, acetylglucosaminyltransferase, voltage-gated K channel, IL-8, ion transport, Map3K8 and Thymidine kinase. In particular, this invention relates to compounds, particularly oligonucleotides, specifically hybridizable with nucleic acids encoding H-Ras, WNT-7B, acetylglucosaminyltransferase, voltage-gated K channel, IL-8, ion transport, Map3K8 and Thymidine kinase polypeptides.

### BACKGROUND OF THE INVENTION

Antisense technology is emerging as an effective means for reducing the expression of specific gene products and may therefore prove to be uniquely useful in a number of therapeutic, diagnostic, and research applications for the modulation of H-Ras, WNT-7B, acetylglucosaminyltransferase, voltage-gated K channel, IL-8, ion transport, Map3K8 and Thymidine kinase.

### SUMMARY OF THE INVENTION

The present invention is directed to compounds, particularly antisense oligonucleotides, which are targeted to a nucleic acid encoding H-Ras, WNT-7B, acetylglucosaminyltransferase, voltage-gated K channel, IL-8, ion transport, Map3K8 or Thymidine kinase, (herein refered to as the “target nucleic acid” or “target nucleic acid sequence”) and which modulate the expression of the target nucleic acid.

In all its various aspects the invention provides an oligonucleotide, e.g., 8-15, 10-25, 10-50 or 20-50 nucleotides in length targeted to a nucleic acid encoding H-Ras, WNT-7B, acetylglucosaminyltransferase, voltage-gated K channel, IL-8, ion transport, Map3K8 or Thymidine kinase. The oligonucleotide hybridizes, e.g., specifically, with the target nucleic acid sequence and inhibits expression the target nucleic acid. The oligonucleotide contains unmodified internucleoside linkages, sugar moieties or nucleotides. Alternatively, the oligonucleotide contains at least one modified internucleoside linkage, sugar moiety or nucleotide.

In one aspect the oligonucleotide is targeted to nucleotides 1-109 or nucleotides 434-513 of a WNT-7B nucleic acid, e.g., SEQ ID NO:1. Alternatively, the oligonucleotide contains at least 10 contiguous nucleotides of SEQ ID NO: 7-11.

In another aspect, the oligonucleotide is targeted to nucleotides 224-366, nucleotides 761-841, or 1062-1142 of a N-acetylglucoaminyltransferase nucleic acid, e.g., SEQ ID NO:2. Alternatively, the oligonucleotide contains at least 10 contiguous nucleotides of SEQ ID NO: 12-16, wherein said oligonucleotide inhibits the expression of N-acetylglucoaminyltransferase.

In a further aspect, the oligonucleotide is targeted to nucleotides 1-121, nucleotides 1226-1201 or 1185-1953 of a voltage gate channel nucleic acid, e.g., SEQ ID NO:3. Alternatively, the oligonucleotide contains at least 10 contiguous nucleotides of SEQ ID NO: 17-21.

In yet a further aspect, the oligonucleotide is targeted to nucleotides 1-91, nucleotides 77-157, nucleotides 902-982 or nucleotides 1541-1621 an ion transport nucleic acid, e.g., SEQ ID NO:4. Alternatively, the oligonucleotide contains at least 10 contiguous nucleotides of the of SEQ ID NO: 23-27.

In one aspect, the oligonucleotide is targeted to nucleotides 63-162 nucleotides 197-246, nucleotides 1037-1186 or nucleotides 1447-1526 of a Map3K8 nucleic acid, e.g., SEQ ID NO:5. Alternatively, the oligonucleotide contains at least 10 contiguous nucleotides of the nucleic acid of SEQ ID NO: 28-32, wherein said oligonucleotide inhibits the expression of Map3K8.

In another aspect, the oligonucleotide is targeted to nucleotides 15-116 nucleotides 132-211, nucleotides 629-708 or nucleotides 1286-1165 of a thymidine kinase nucleic acid, e.g., SEQ ID NO:6. Alternatively, the oligonucleotide contains at least 10 contiguous nucleotides of SEQ ID NO: 33-37, 18.

Further provided are methods of modulating, e.g., inhibiting or increasing the expression of H-Ras, WNT-7B, acetylglucosaminyltransferase, voltage-gated K channel, IL-8, ion transport, Map3K8 and Thymidine kinase in a cell or tissues by contacting the cells or tissue with one or more of the antisense compounds or compositions of the invention.

Also provided are methods of inhibiting cell proliferation, by contacting a cell with one or more of the WNT-7B or acetylglucosaminyltransferase antisense compounds or compositions of the invention.

The invention further provides a method of increasing the production, e.g., secretion, of Il-1b in a cell by contacting a cell with one or more of thymidine kinase antisense compounds or compositions of the invention.

The oligonucleotide is present at a concentration of 1 mM, 5 mM, 10 mM, 25 mM, 50 mM or greater. Preferably the oligonucleotide is present at a concentration of 400 mM. The cell is for a example a lymphoid cell, a stem cell, a blood cell, an epithelial cell, an endothelial cell, an ovarian cell, or a tumor cell.

Pharmaceutical and other compositions comprising the compounds of the invention are also provided. Further provided are methods of treating an animal, particularly a human, suspected of having or being prone to a disease or condition associated with expression of H-Ras, WNT-7B, acetylglucosaminyltransferase, voltage-gated K channel, IL-8, ion transport, Map3K8 or Thymidine kinase by administering a therapeutically or prophylactically effective amount of one or more of the antisense compounds or compositions of the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### BRIEF DESCRIPTION OF THE DRAWINGS

FIG. 1 is a schematic representation of various nucleic acid structures present in mixed backbone nucleic acids. FIG. 1A shows backbone structures for DNA, S-DNA, peptide nucleic acids (PNA), morpholino, and 5′-methoxyethyl nucleic acids. FIG. 1B shows sugar structures for (1) unmodified RNA, (2) 2′-O-methyl RNA, (3) 2′-amino RNA, (4) 2′-C-allyl RNA, and a (5) 3′-3′-inverted thymidine nucleoside.

FIG. 2 is a series of bar graphs showing relative expression of different genes under various antisense (AS) conditions compared to scatter control (SC) or control (C) conditions measured using TaqMan analysis: FIG. 2A, H-Ras; FIG. 2B, acetylglucoaminotransferase SW60 cells and LX-1 cells; FIG. 2C, Wnt-7B; FIG. 2D, Thymidine kinase; FIG. 2E, Ion Channel (Ag1987); FIG. 2F, interleukin-8; FIG. 2G, the Map3K8 like (Ag3116).

FIG. 3 is a series of bar graphs showing how cellular proliferation is affected by various concentrations of antisense (AS) nucleic acids, scatter control (SC) or control (CTR) nucleic acid. FIG. 3A, T-24 cell proliferation with H-ras antisense (RAS); FIG. 3B, SW620 cell proliferation with acetylglucoaminyltransferase antisense; FIG. 3C, LX-1 cell proliferation with acetylglucoaminyltransferase antisense; FIG. 3D, SW620 cell proliferation with acetylglucoaminyltransferase antisense; FIG. 3E, NC1-H460 cell proliferation with acetylglucoaminyltransferase antisense.

FIGS. 4A and 4B are bar graphs indicating cellular proliferation in the presence of various concentrations of Wnt-7B antisense (AS) nucleic acids, scatter control (SC) or control (CTR) nucleic acids: MDA-MB-468 cell proliferation (FIG. 4A) and MCF-7 cell proliferation (FIG. 4B).

FIG. 5 is two bar graphs showing changes in secretion of protein via ELISA assay due to treatment with antisense nucleic acids in THP-1 cells. FIG. 5A shows secretion of interleukin-8 (IL-8) with antisense (AS) nucleic acids specific for IL-8 compared to scatter control (SC) and control (CTR). FIG. 5B shows the changes in the secretion of interleukin-1β in response to antisense nucleic acids specific for thymidine kinase.

FIG. 6 is a Western immunoblot of lamin A/C in Hela-S3 cells and p53 in SW-620 cells with increasing concentrations of mixed backbone (M-B) antisense DNA or small interfering RNA (siRNA).

FIG. 7 is a Western immunoblot of GAPDH and TS in SW-620 cells with varying concentrations of mixed backbone (M-B) antisense DNA or small interfering RNA (siRNA).

FIG. 8 is two graphs showing changes in lamin A/C mRNA measured using TaqMan with varying concentrations of antisense or interfering nucleic acids: FIG. 8A, mixed backbone (M-B) antisense DNA; FIG. 8B, small interfering RNA (siRNA).

FIG. 9 is two graphs showing changes in TS mRNA due to varying concentrations of interfering or antisense nucleic acids in Hela-S3 cells: FIG. 9A. siRNA. FIG. 9B, M-B antisense DNA.

FIG. 10 is two graphs showing changes in p53 mRNA due to varying concentrations of interfering or antisense nucleic acids in cells: FIG. 10A, siRNA; FIG. 10B, M-B antisense DNA.

FIG. 11 is a graph showing fluorescence activated cell sorting analysis of the reduction of the number of cells expressing MHC Class I in response to antisense for Ion Channel.

### DETAILED DESCRIPTION

The present invention employs oligomeric compounds, particularly antisense oligonucleotides and small interfering RNA (siRNA), for use in modulating the function, e.g., expression of nucleic acid molecules encoding H-Ras, WNT-7B, acetylglucosaminyltransferase, voltage-gated K channel, IL-8, ion transport, Map3K8 and Thymidine kinase and modulating the amount of these proteins produced. In addition, the invention relates to inhibiting cell proliferation by modulating the function of oncology targets; H-Ras, WNT-7B, and acetylglucosaminyltransferase. This is accomplished by providing antisense compounds and siRNA which specifically hybridize with one or more nucleic acids encoding H-Ras, WNT-7B, acetylglucosaminyltransferase, voltage-gated K channel, IL-8, ion transport, Map3K8 and Thymidine kinase. As used herein, the terms “target nucleic acid” and “nucleic acid encoding H-Ras, WNT-7B, acetylglucosaminyltransferase, voltage-gated K channel, IL-8, ion transport, Map3K8 and Thymidine kinase “encompass DNA encoding H-Ras, WNT-7B, acetylglucosaminyltransferase, voltage-gated K channel, IL-8, ion transport, Map3K8 and Thymidine kinase, RNA (including pre-mRNA and mRNA) transcribed from such DNA, and also cDNA derived from such RNA. The specific hybridization of an oligomeric compound with its target nucleic acid interferes with the normal function of the nucleic acid. This modulation of function of a target nucleic acid by compounds which specifically hybridize to it is generally referred to as “antisense”. Additionally, modulation of function of a target nucleic acid is also accomplished by siRNA. siRNAs inhibit gene expression by inducing RNAi. siRNAs are 21- to 23-nucleotide RNA particles, with characteristic 2- to 3-nucleotide 3′-overhanging ends, which are generated by ribonuclease III cleavage from longer dsRNAs.

The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of nucleic acid encoding H-Ras, WNT-7B, acetylglucosaminyltransferase, voltage-gated K channel, IL-8, ion transport, Map3K8 or Thymidine kinase. In the context of the present invention, “modulation” means either an increase (stimulation) or a decrease (inhibition) in the expression of a gene. In the context of the present invention, inhibition is the preferred form of modulation of gene expression and mRNA is a preferred target.

Antisense modulation of H-Ras, WNT-7B, acetylglucosaminyltransferase, voltage-gated K channel, IL-8, ion transport, Map3K8 or Thymidine kinase expression can be assayed in a variety of ways known in the art. For example, H-Ras, WNT-7B, acetylglucosaminyltransferase, voltage-gated K channel, IL-8, ion transport, Map3K8 or Thymidine kinase −1 mRNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+ mRNA. Methods of RNA isolation are taught in, for example, Ausubel, F. M. et al., Current Protocols in Molecular Biology, Volume 1, pp. 4.1.1-4.2.9 and 4.5.1-4.5.3, John Wiley & Sons, Inc., 1993. Northern blot analysis is routine in the art and is taught in, for example, Ausubel, F. M. et al., Current Protocols in Molecular Biology, Volume 1, pp. 4.2.1-4.2.9, John Wiley & Sons, Inc., 1996. Real-time quantitative (PCR) can be conveniently accomplished using the commercially available ABI PRISM.™. 7700 Sequence Detection System, available from PE-Applied Biosystems, Foster City, Calif. and used according to manufacturer's instructions.

Protein levels of H-Ras, WNT-7B, acetylglucosaminyltransferase, voltage-gated K channel, IL-8, ion transport, Map3K8 or Thymidine kinase can be quantitated in a variety of ways well known in the art, such as immunoprecipitation, Western blot analysis (immunoblotting), ELISA or fluorescence-activated cell sorting (FACS). Antibodies directed to H-Ras, WNT-7B, acetylglucosaminyltransferase, voltage-gated K channel, IL-8, ion transport, Map3K8 or Thymidine kinase can be identified and obtained from a variety of sources, such as the MSRS catalog of antibodies (Aerie Corporation, Birmingham, Mich.), or can be prepared via conventional antibody generation methods. Methods for preparation of polyclonal antisera are taught in, for example, Ausubel, F. M. et al., Current Protocols in Molecular Biology, Volume 2, pp. 11.12.1-11.12.9, John Wiley & Sons, Inc., 1997. Preparation of monoclonal antibodies is taught in, for example, Ausubel, F. M. et al., Current Protocols in Molecular Biology, Volume 2, pp. 11.4.1-11.11.5, John Wiley & Sons, Inc., 1997.

Immunoprecipitation methods are standard in the art and can be found at, for example, Ausubel, F. M. et al., Current Protocols in Molecular Biology, Volume 2, pp. 10.16.1-10.16.11, John Wiley & Sons, Inc., 1998. Western blot (immunoblot) analysis is standard in the art and can be found at, for example, Ausubel, F. M. et al., Current Protocols in Molecular Biology, Volume 2, pp. 10.8.1-10.8.21, John Wiley & Sons, Inc., 1997. Enzyme-linked immunosorbent assays (ELISA) are standard in the art and can be found at, for example, Ausubel, F. M. et al., Current Protocols in Molecular Biology, Volume 2, pp. 11.2.1-11.2.22, John Wiley & Sons, Inc., 1991.

It is preferred to target specific nucleic acids for antisense or siRNA. “Targeting” an antisense compound to a particular nucleic acid, in the context of this invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding H-Ras, WNT-7B, acetylglucosaminyltransferase, voltage-gated K channel, IL-8, ion transport, Map3K8 or Thymidine kinase. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding H-Ras, WNT-7B, acetylglucosaminyltransferase, voltage-gated K channel, IL-8, ion transport, Map3K8 or Thymidine kinase, regardless of the sequence(s) of such codons. It is also known in the art that a translation termination codon (or “stopcodon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and” translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a preferred target region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence mRNA splice sites, i.e., intron-exon junctions, may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

Once one or more target sites have been identified, oligonucleotides are chosen which are sufficiently complementary to the target, i.e., hybridize sufficiently well and with sufficient specificity, to give the desired effect.

In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleotides which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an antisense compound or siRN need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

Antisense, siRNA, and other compounds of the invention which hybridize to the target and inhibit expression of the target are identified through experimentation, and the sequences of these compounds are herein below identified as preferred embodiments of the invention. The target sites to which these preferred sequences are complementary are herein below referred to as “active sites” and are therefore preferred sites for targeting. Therefore another embodiment of the invention encompasses compounds which hybridize to these active sites.

Antisense compounds are commonly used as research reagents and diagnostics. For example, antisense oligonucleotides, which are able to inhibit gene expression with exquisite specificity, are often used by those of ordinary skill to elucidate the function of particular genes. Antisense compounds are also used, for example, to distinguish between functions of various members of a biological pathway. Antisense modulation has, therefore, been harnessed for research use.

The specificity and sensitivity of antisense is also harnessed by those of skilled in the art for therapeutic uses. Antisense oligonucleotides have been employed as therapeutic moieties in the treatment of disease states in animals and man. Antisense oligonucleotide drugs, including ribozymes, have been safely and effectively administered to humans and numerous clinical trials are presently underway. It is thus established that oligonucleotides can be useful therapeutic modalities that can be configured to be useful in treatment regimes for treatment of cells, tissues and animals, especially humans.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleotides, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 50 nucleotides (i.e. from about 8 to about 50 linked nucleosides). Particularly preferred antisense compounds are antisense oligonucleotides, even more preferably those comprising from about 12 to about 30 nucleotides. Antisense compounds include ribozymes, external guide sequence(EGS) oligonucleotides (oligozymes), and other short catalytic RNAs or catalytic oligonucleotides which hybridize to the target nucleic acid and modulate its expression.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone can also be considered to be oligonucleosides.

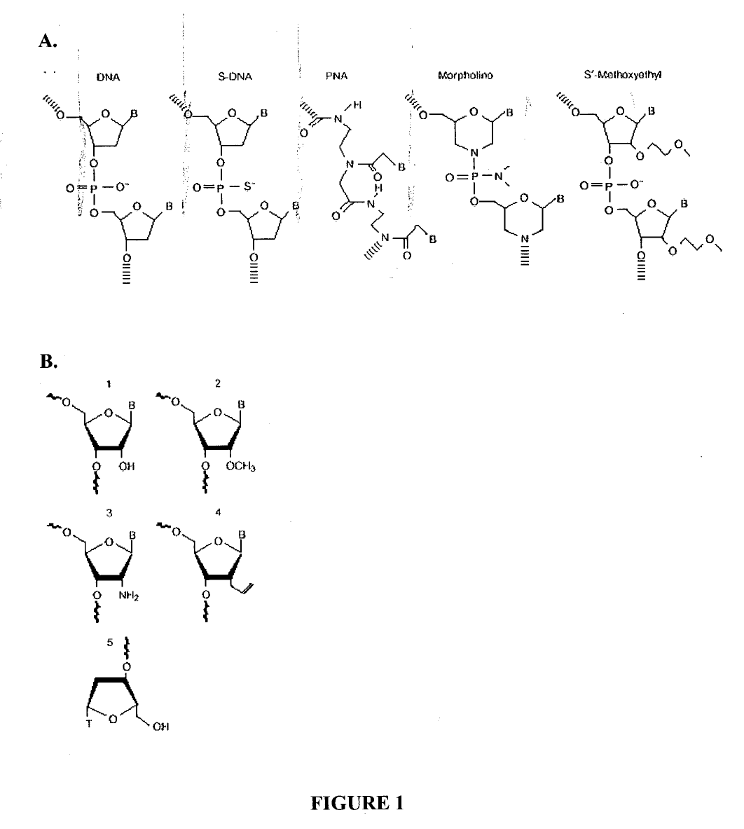

Preferred modified oligonucleotide backbones include, for example, phosphorothioates, chiral phosphorothioates, phosphorodithioates, phosphotriesters, aminoalkylphosphotriesters, methyl and other alkylphosphonates including 3′-alkylene phosphonates, 5′-alkylene phosphonates and chiral phosphonates, phosphinates, phosphoramidates including 3′-aminophosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3-most internucleotide linkage i.e. a single inverted nucleoside residue which may be a basic (the nucleobase is missing or has a hydroxyl group in place thereof). Exemplary modified oligonuceotide bases are illustrated in FIG. 1. Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholinolinkages (formed in part from the sugar portion of a nucleoside); siloxanebackbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, each of which is herein incorporated by reference.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleotides are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Most preferred embodiments of the invention are oligonucleotides with phosphorothioate backbones and oligonucleosides with hetero atom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH.sub.3)—N(CH₃)—CH₂—and—O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O [(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃,OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78,486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples hereinbelow.

A further preferred modification includes Locked Nucleic Acids (LNAs) in which the 2′-hydroxyl group is linked to the 3′ or 4′ carbon atom of the sugar ring thereby forming a bicyclic sugar moiety. The linkage is preferably a methelyne (—CH₂—)ₙ group bridging the 2′ oxygen atom and the 3′ or 4′ carbon atom wherein n is 1 or 2. LNAs and preparation thereof are described in WO98/39352 and WO 99/14226.

Other preferred modifications include 2′-methoxy (2′-O—CH₃), 2′-aminopropoxy (2′-OCH₂CH₂CH₂NH₂), 2′-allyl (2′-CH₂—CH.dbd.CH₂), 2′-O-allyl (2′-O—CH₂—CH.dbd.CH₂) and 2′-fluoro(2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, each of which is herein incorporated by reference in its entirety.

Oligonucleotides may also include nucleobase (often referred to in the art simply as “base”) modifications or substitutions. As used herein, “unmodified” or “natural” nucleotides include the purine bases adenine (A) and guanine (G), and the pyrimidine bases thymine (T), cytosine (C) and uracil (U). Modified nucleotides include other synthetic and natural nucleotides such as5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil,2-thiothymine and 2-thiocytosine, 5-halouracil and cytosine, 5-propynyl(-C.ident.C—CH₃) uracil and cytosine and other alkynyl derivatives of pyrimidine bases, 6-azo uracil, cytosine and thymine, 5-uracil (pseudo uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanineand 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleotides include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazolecytidine (2H-pyrimido[4,5-b]indol-2-one), pyridoindole cytidine(H-pyrido[3′,2′:4,5]pyrrolo[2,3-d]pyrimidin-2-one). Modified nucleotides may also include those in which the purine or pyrimidine base is replaced with other hetero cycles, for example 7-deaza-adenine, 7-deazaguanosine, 2 aminopyridine and 2-pyridone. Further nucleotides include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990,those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B. ed., CRC Press, 1993. Certain of these nucleotides are particularly useful for increasing the binding affinity of the oligomeric compounds of the invention. These include 5-substituted pyrimidines, 6-azapyrimidines and N-2, N-6 and 0-6 substituted purines, including 2-aminopropyladenine, 5-propynyluracil and S-propynylcytosine. 5-methylcytosine substitutions have been shown to increase nucleic acid duplex stability by 0.6-1.2.degree. C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleotides as well as other modified nucleotides include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941, each of which is herein incorporated by reference, and U.S. Pat. No. 5,750,692, also herein incorporated by reference.

Another modification of the oligonucleotides of the invention involve schemically linking to the oligonucleotide one or more moieties or conjugates which enhance the activity, cellular distribution or cellular uptake of the oligonucleotide. The compounds of the invention can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Representative conjugate groups are disclosed in International Patent Application PCT/US92/09196, filed Oct. 23, 1992 the entire disclosure of which is incorporated herein by reference. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), athiocholesterol (Oberhauser et al., Nucl. Acids Res., 1992, 20, 533-538), analiphatic chain, e.g., dodecandiol or undecyl residues (Saison-Behmoaras et al., EMBO J., 1991, 10, 1111-1118; Kabanov et al., FEBS Lett., 1990, 259, 327-330;Svinarchuk et al., Biochimie, 1993, 75, 49-54), a phospholipid, e.g., di-hexadecyl-rac-glycerol or triethyl-ammonium1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides &Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine orhexylamino-carbonyl-oxycholesterol moiety (Crooke et al., J. Pharmacol. Exp. Ther., 1996, 277, 923-937. Oligonucleotides of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, abarbiturate, a cephalosporin, a sulfa drug, an antidiabetic, an antibacterial or an antibiotic.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

It is not necessary for all positions in a given compound to be uniformly modified, and in fact more than one of the aforementioned modifications may be incorporated in a single compound or even at a single nucleoside within an oligonucleotide. The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The antisense compounds of the invention are synthesized in vitro and do not include antisense compositions of biological origin, or genetic vector constructs designed to direct the in vivo synthesis of antisense molecules. The compounds of the invention may also be mixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds of the invention encompass any pharmaceutically acceptable salts; esters, or salts of such esters, or any other compound which, upon administration to an animal including a human, is capable of providing(directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells there of by the action of endogenous enzymes or other chemicals and/or conditions. In particular, prodrug versions of the oligonucleotides of the invention are prepared as SATE [(S-acetyl-2-thioethyl) phosphate] derivatives according to the methods disclosed in WO 93/24510, published Dec. 9, 1993 or in WO 94/26764 and U.S. Pat. No. 5,770,713

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci., 1977, 66, 1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonicacid, benzenesulfonic acid, 4-methylbenzenesulfonic acid, naphthalene-2-sulfonicacid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The antisense compounds of the present invention can be utilized for diagnostics, therapeutics, prophylaxis and as research reagents and kits. For therapeutics, an animal, preferably a human, suspected of having a disease or disorder which can be treated by modulating the expression of H-Ras, WNT-7B, acetylglucosaminyl transferase, voltage-gated K channel, IL-8, ion transport, Map3K8 or Thymidine kinase is treated by administering antisense compounds in accordance with this invention. The compounds of the invention can be utilized in pharmaceutical compositions by adding an effective amount of an antisense compound to a suitable pharmaceutically acceptable diluent or carrier. Use of the antisense compounds and methods of the invention may also be useful prophylactically, e.g., to prevent or delay infection, inflammation, tumor formation or growth, or tumor metastasis for example.

The antisense compounds of the invention are useful for research and diagnostics, because these compounds hybridize to nucleic acids encoding H-Ras, WNT-7B, acetylglucosaminyl transferase, voltage-gated K channel, IL-8, ion transport, Map3K8 or Thymidine kinase, enabling sandwich and other assays to easily be constructed to exploit this fact. Hybridization of the antisense oligonucleotides of the invention with a nucleic acid encoding H-Ras, WNT-7B, acetylglucosaminyl transferase, voltage-gated K channel, IL-8, ion transport, Map3K8 or Thymidine kinase can be detected by means known in the art. Such means may include conjugation of an enzyme to the oligonucleotide, radiolabelling of the oligonucleotide or any other suitable detection means. Kits using such detection means for detecting the level of H-Ras, WNT-7B, acetylglucosaminyl transferase, voltage-gated K channel, IL-8, ion transport, Map3K8 or Thymidine kinase in a sample may also be prepared.

The present invention also includes pharmaceutical compositions and formulations which include the antisense compounds of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the oligonucleotides of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP anddioleoylphosphatidyl ethanolamine DOTMA). Oligonucleotides of the invention maybe encapsulated within liposomes or may form complexes thereto, in particular to cationic liposomes. Alternatively, oligonucleotides may be complexed to lipids, in particular to cationic lipids. Preferred fatty acids and esters include but are not limited arachidonic acid, oleic acid, eicosanoic acid, lauric acid, caprylic acid, capric acid, myristic acid, palmitic acid, stearic acid, linoleicacid, linolenic acid, dicaprate, tricaprate, monoolein, dilaurin, glyceryl 1-monocaprate, 1-dodecylazacycloheptan-2-one, an acylcarnitine, an acylcholine, or a C₁₋₁₀ alkyl ester (e.g. isopropylmyristate IPM), monoglyceride, diglyceride or pharmaceutically acceptable salt thereof.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which oligonucleotides of the invention are administered in conjunction with one or more penetration enhancers surfactants and chelators. Preferred surfactants include fatty acids and/or esters or salts thereof, bile acids and/or salts thereof. Preferred bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate, sodium glycodihydrofusidate. Preferred fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or amonoglyceride, a diglyceride or a pharmaceutically acceptable salt thereof (e.g. sodium). Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Oligonucleotides of the invention may be delivered orally in granular form including sprayed dried particles, or complexed to form micro or nanoparticles. Oligonucleotide complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Particularly preferred complexing agents include chitosan, N-trimethylchitosan, poly-L-lysine, polyhistidine, polyornithine, polyspermines, protamine, polyvinylpyridine, polythiodiethylaminomethylethylene P(TDAE), polyaminostyrene (e.g. p-amino), poly(methylcyanoacrylate), poly(ethylcyanoacrylate),poly(butylcyanoacrylate), poly(isobutylcyanoacrylate),poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid(PLGA), alginate, and polyethyleneglycol (PEG).

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

In one embodiment of the present invention the pharmaceutical compositions maybe formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention.

Emulsions

The compositions of the present invention may be prepared and formulated as emulsions. Emulsions are typically heterogenous systems of one liquid dispersed in another in the form of droplets usually exceeding 0.1 μm in diameter. (Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising of two immiscible liquid phases intimately mixed and dispersed with each other. In general, emulsions may be either water-in-oil (w/o) or of the oil-in-water (o/w) variety. When an aqueous phase is finely divided into and dispersed as minute droplets into a bulk oily phase the resulting composition is called a water-in-oil (w/o) emulsion. Alternatively, when an oily phase is finely divided into and dispersed as minute droplets into a bulk aqueous phase the resulting composition is called an oil-in-water (o/w) emulsion. Emulsions may contain additional components in addition to the dispersed phases and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous provides an o/w/o emulsion.

Emulsions are characterized by little or no hermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (Rieger, in Pharmaceutical Dosage Forms, Lieberman, Rieger and Banker(Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group:nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases posess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, non swelling clays such as bentonite, attapulgite, hectorite, kaolin, montmorillonite, colloidal aluminum silicate and colloidal magnesium aluminum silicate, pigments istearate.

A large variety of non-emulsifying materials are also included in emulsion formulations and contribute to the properties of emulsions. These include fats, oils, waxes, fatty acids, fatty alcohols, fatty esters, humectants, hydrophiliccolloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginicacid, carrageenan, guar gum, karaya gum, and tragacanth), cellulose derivatives(for example, carboxymethyl cellulose and carboxypropyl cellulose), and syntheticpolymers (for example, carbomers, cellulose ethers, and carboxyvinyl polymers).These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propylparaben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of reasons of ease of formulation, efficacy from an absorption and bioavailability standpoint. (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment of the present invention, the compositions of oligonucleotides and nucleic acids are formulated as microemulsions. A microemulsion may be defined as a system of water, oil and amphiphile which is a single optically isotropic and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335).Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethyleneoleyl ethers, polyglycerol fatty acid esters, tetraglycerol monolaurate (ML310),tetraglycerol monooleate (MO310), hexaglycerol monooleate (PO₃₁₀), hexaglycerol pentaoleate (PO500), decaglycerol monocaprate (MCA750), decaglycerol monooleate (MO750), decaglycerol sequioleate (SO750), decaglycerol decaoleate (DAO750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono, di, and triglycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994,11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al., J. Pharm. Sci., 1996, 85, 138-143). Often microemulsions may form spontaneously when their components are brought together at ambient temperature. This may be particularly advantageous when formulating thermolabile drugs, peptides or oligonucleotides. Microemulsions have also been effective in the transdermal delivery of active components in both cosmetic and pharmaceutical applications. It is expected that the microemulsion compositions and formulations of the present invention will facilitate the increased systemic absorption of oligonucleotides and nucleic acids from the gastrointestinal tract, as well as improve the local cellular uptake of oligonucleotides and nucleic acids within the gastrointestinal tract, vagina, buccal cavity and other areas of administration.

Microemulsions of the present invention may also contain additional components and additives such as sorbitan monostearate (Grill 3), Labrasol, and penetration enhancers to improve the properties of the formulation and to enhance the absorption of the oligonucleotides and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

Liposomes

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in aspherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

In order to cross intact mammalian skin, lipid vesicles must pass through a series of fine pores, each with a diameter less than 50 nm, under the influence of a suitable transdermal gradient. Therefore, it is desirable to use a liposome which is highly deformable and able to pass through such fine pores.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes. As the merging of the liposome and cell progresses, the liposomal contents are emptied into the cell where the active agent may act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis.

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC)such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g. as a solution or as an emulsion) were ineffective (Weiner et al., Journal of Drug Targeting, 1992, 2, 405-410).Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18,259-265).

Non-ionic liposomal systems have also been examined to determine their utility in the delivery of drugs to the skin, in particular systems comprising non-ionicsurfactant and cholesterol. Non-ionic liposomal formulations comprising Novasome™ (glyceryl dilaurate/cholesterol/polyoxyethylene-10-stearyl ether)and Novasome™ II (glyceryl distearate/cholesterol/polyoxyethylene-10-stearylether) were used to deliver cyclosporin-A into the dermis of mouse skin. Results indicated that such non-ionic liposomal systems were effective in facilitating the deposition of cyclosporin-A into different layers of the skin (Hu et al. S. T. P. Pharma. Sci., 1994, 4, 6, 466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Various liposomes comprising one or more glycolipids are known in the art. Papahadjopoulos et al. (Ann. N.Y. Acad. Sci., 1987, 507, 64) reported the ability of monosialoganglioside, galactocerebroside sulfate and phosphatidylinositol to improve blood half-lives of liposomes.

Many liposomes comprising lipids derivatized with one or more hydrophilicpolymers, and methods of preparation thereof, are known in the art. Sunamoto et al. (bull. Chem. Soc. Jpn., 1980, 53, 2778) described liposomes comprising anonionic detergent, that contains a PEG moiety. Illum et al. (febs lett., 1984, 167, 79) noted that hydrophilic coating of polystyreneparticles with polymeric glycols results in significantly enhanced bloodhalf-lives. Synthetic phospholipids modified by the attachment of carboxylicgroups of polyalkylene glycols (e.g., PEG) are described by sears (U.S. Pat. Nos. 4,426,330 and 4,534,899). Klibanov et al. (Febs lett., 1990, 268, 235) described experiments demonstrating that liposomes comprising phosphatidylethanolamine (PE) derivatized with PEG or PEG stearate have significant increases in blood circulation half-lives.

Transfersomes are yet another type of liposomes, and are highly deformable lipidaggregates which are attractive candidates for drug delivery vehicles. Transfersomes may be described as lipid droplets which are so highly deformablethat they are easily able to penetrate through pores which are smaller than the droplet. Transfersomes are adaptable to the environment in which they are used, e.g. they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition.transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (hlb). The nature of the hydrophilic group (also known as the “head”) provides the most useful means for categorizing the different surfactants used in formulations (Rieger, In Pharmaceutical Dosage Forms, Marcel Dekker, Inc., New York, N.Y., 1988, p.285).

If the surfactant molecule is not ionized, it is classified as a nonionic surfactant. Nonionic surfactants find wide application in pharmaceutical and cosmetic products and are usable over a wide range of pH values. In general their hlb values range from 2 to about 18 depending on their structure. Non ionic surfactants include nonionic esters such as ethylene glycol esters, propyleneglycol esters, glyceryl esters, polyglyceryl esters, sorbitan esters, sucro seesters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactantsinclude carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl tauratesand sulfosuccinates, and phosphates. The most important members of the anionicsurfactant class are the alkyl sulfates and the soaps.

If the surfactant molecule carries a positive charge when it is dissolved ordispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, n-alkylbetaines and phosphatides.

The use of surfactants in drug products, formulations and in emulsions has been reviewed (Rieger, In Pharmaceutical Dosage Forms, Marcel Dekker, Inc., New York, N.Y., 1988, p. 285).

Penetration Enhancers

In one embodiment, the present invention employs various penetration enhancers to effect the efficient delivery of nucleic acids, particularly oligonucleotides, to the skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers may be classified as belonging to one of five broad categories, i.e., surfactants, fatty acids, bile salts, chelating agents, andnon-chelating non-surfactants (Lee et al., Critical Reviews In Therapeutic Drug Carrier Systems, 1991, p.92). Each of the above mentioned classes of penetration enhancers are described below in greater detail.

Surfactants: In connection with the present invention, surfactants (or “surface-active agents”) are chemical entities which, when dissolved in an aqueous solution, reduce the surface tension of the solution or the interfacial tension between the aqueous solution and another liquid, with the result that absorption of oligonucleotides through the mucosa is enhanced. In addition to bile salts and fatty acids, these penetration enhancers include, for example, sodium lauryl sulfate, polyoxyethylene-9-lauryl ether and polyoxyethylene-20-cetyl ether) (Lee et al., Critical Reviews In Therapeutic Drug Carrier Systems, 1991, p.92); and perfluorochemical emulsions, such asfc-43. Takahashi et al., J. Pharm. Pharmacol., 1988, 40, 252).

Fatty acids: various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcamitines, acylcholines, C₁₋₁₀ alkylesters thereof (e.g., methyl, isopropyl and t-butyl), and mono- and di-glycerides thereof (i.e., oleate, laurate, caprate, myristate, palmitate, stearate, linoleate, etc.) (Lee et al., Critical Reviews In Therapeutic Drug Carrier Systems, 1991, p.92; Muranishi, Critical Reviews In Therapeutic Drug Carrier Systems, 1990, 7, 1-33; El Hariri et al., J. Pharm. Pharmacol., 1992,44, 651-654).

Bile salts: the physiological role of bile includes the facilitation of dispersion and absorption of lipids and fat-soluble vitamins (Brunton, chapter 38 in: Goodman & Gilman's the Pharmacological Basis of Therapeutics, 9th ed., Hardman et al. Eds., Mcgraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. The bile salts of the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodiumtaurodeoxycholate), chenodeoxycholic acid (sodium chenodeoxycholate), ursodeoxycholic acid (UDCA), sodium tauro-24,25-dihydro-fusidate (STDHF), sodiumglycodihydrofusidate and polyoxyethylene-9-lauryl ether (POE).

Chelating agents: chelating agents, as used in connection with the present invention, can be defined as compounds that remove metallic ions from solution by forming complexes therewith, with the result that absorption of oligonucleotides through the mucosa is enhanced. With regards to their use as penetration enhancers in the present invention, chelating agents have the added advantage of also serving as dnase inhibitors, as most characterized nucleases require a divalent metal ion for catalysis and are thus inhibited by chelating agents (Jarrett, J. Chromatogr., 1993, 618, 315-339). Chelating agentsof the invention include but are not limited to disodiumethylenediaminetetraacetate (EDTA), citric acid, salicylates (e.g., sodiumsalicylate, 5-methoxysalicylate and homovanilate), n-acyl derivatives of collagen, laureth-9 and n-amino acyl derivatives of beta-diketones(enamines) (Lee et al., Critical Reviews In Therapeutic Drug Carrier Systems, 1991, page 92; Muranishi, Critical Reviews In Therapeutic Drug Carrier Systems,1990, 7, 1-33; Buur et al., J. Control rel., 1990, 14, 43-51).

Non-chelating non-surfactants: as used herein, non-chelating non-surfactant penetration enhancing compounds can be defined as compounds that demonstrate insignificant activity as chelating agents or as surfactants but that nonetheless enhance absorption of oligonucleotides through the alimentary mucosa. This class of penetration enhancers include, for example, unsaturated cyclic ureas, 1-alkyl- and 1-alkenylazacyclo-alkanone derivatives; and non-steroidal anti-inflammatory agents such as diclofenac sodium, indomethacin and phenylbutazone (Yamashita et al., J. Pharm. Pharmacol., 1987, 39, 621-626)

Agents that enhance uptake of oligonucleotides at the cellular level may also be added to the pharmaceutical and other compositions of the present invention. Forexample, cationic lipids, such as lipofectin (Junichi et al, U.S. Pat. No. 5,705,188), cationic glycerol derivatives, and polycationic molecules, such as polylysine, are also known to enhance the cellular uptake of oligonucleotides.

Other agents may be utilized to enhance the penetration of the administered nucleic acids, including glycols such as ethylene glycol and propylene glycol, pyrrols such as 2-pyrrol, azones, and terpenes such as limonene and menthone.

Carriers

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate oligonucleotide in hepatic tissue can be reduced when it is coadministered with polyinosinic acid, dextran sulfate, polycytidic acid or 4-acetamido-4′isothiocyano-stilbene-2,2′-disulfonic acid (Miyao et al., Antisense Res. Dev., 1995, 5, 115-121; Takakura et al., Antisense & Nucl. Acid Drug Dev., 1996, 6,177-183).

Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodiumlauryl sulphate, etc.).

Pharmaceutically acceptable organic or inorganic excipient suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethyl cellulose, polyvinylpyrrolidone and the like.

Other Components

The compositions of the present invention may additionally contain other adjunct components conventionally found in pharmaceutical compositions, at their art-established usage levels. Thus, for example, the compositions may containadditional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and. stabilizers. However, such materials, when added, should not unduly interferewith the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances which increase the viscosity of the suspension including, for example, sodium carboxymethylcellulose, sorbitol and/or dextran. The suspension may also contain stabilizers.

Certain embodiments of the invention provide pharmaceutical compositions containing (a) one or more antisense compounds and (b) one or more other chemotherapeutic agents which function by a non-antisense mechanism. Examples of such chemotherapeutic agents include but are not limited to daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin c, actinomycin d, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil(5-fu), 5-fluorodeoxyuridine (5-fudr), methotrexate (mtx), colchicine, taxol, vincristine, vinblastine, etoposide (vp-16), trimetrexate, irinotecan, topotecan, gemcitabine, teniposide, cisplatin and diethylstilbestrol (des). See, generally, the Merck Manual of Diagnosis and Therapy, 15th ed. 1987, pp.1206-1²²⁸, Berkow et al., eds., Rahway, N.J. when used with the compounds of the invention, such chemotherapeutic agents may be used individually (e.g., 5-fu and oligonucleotide), sequentially (e.g., 5-fu and oligonucleotide for a period of time followed by mtx and oligonucleotide), or in combination with one or moreother such chemotherapeutic agents (e.g., 5-fu, mtx and oligonucleotide, or 5-fu, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovirand ganciclovir, may also be combined in compositions of the invention. See, generally, the Merck Manual of Diagnosis and Therapy, 15th ed., Berkow et al.,eds., 1987, Rahway, N.J., pages 2499-2506 and 46-49, respectively). Other non-antisense chemotherapeutic agents are also within the scope of this invention. Two or more combined compounds may be used together or sequentially.

In another related embodiment, compositions of the invention may contain one or more antisense compounds, particularly oligonucleotides, targeted to a first nucleic acid and one or more additional antisense compounds targeted to a second nucleic acid target. Numerous examples of antisense compounds are known in the art. Two or more combined compounds may be used together or sequentially.

The formulation of therapeutic compositions and their subsequent administrationis believed to be within the skill of those in the art. Dosing is dependent on severity and responsiveness of the disease state to be treated, with the courseof treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on ec.sub.50 s found to be effective in in vitro and in vivo animal models. In general, dosage is from 0.01 ug to 100 g per kg of bodyweight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 ug to 100 g per kg of bodyweight, once or more daily, to once every 20 years.

The invention will be further described in the following examples, which do not limit the scope of the invention described in the claims.

### EXAMPLES

### Example 1

### General Methods

Quantitative Expression of Target mRNA

The quantitative expression of various clones was assessed using microtiter plates containing RNA samples from a variety of cells, cell lines and tissues using real time quantitative PCR (RTQ PCR). RTQ PCR was performed on an Applied Biosystems ABI PRISM® 7700 or an ABI PRISM® 7900 HT Sequence Detection System.

RNA integrity from all samples was controlled for quality by visual assessment of agarose gel electropherograms using 28S and 18S ribosomal RNA staining intensity ratio as a guide (2:1 to 2.5:1 28s:18s) and the presence or absence of low molecular weight RNAs which areindicative of degradation products. Samples are controlled against genomic DNA contamination by RTQ PCR reactions run in the absence of reverse transcriptase using probe and primer sets designed to amplify across the span of a single exon.

The RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples were then normalized to reference nucleic acids, using 1×TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primer: primer concentration=250 nM, primer melting temperature (Tm) range=58°-60° C., primer optimal Tm=59° C., maximum primer difference=2° C., probe does not have 5′G, probe Tm must be 10° C. greater than primer Tm, amplicon size 75 bp to 100 bp. The probes and primer selected were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: For RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as ! follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described for RNA samples. PCR reactions containing one or two sets of probe and primers were set up as described, using 1×TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute.

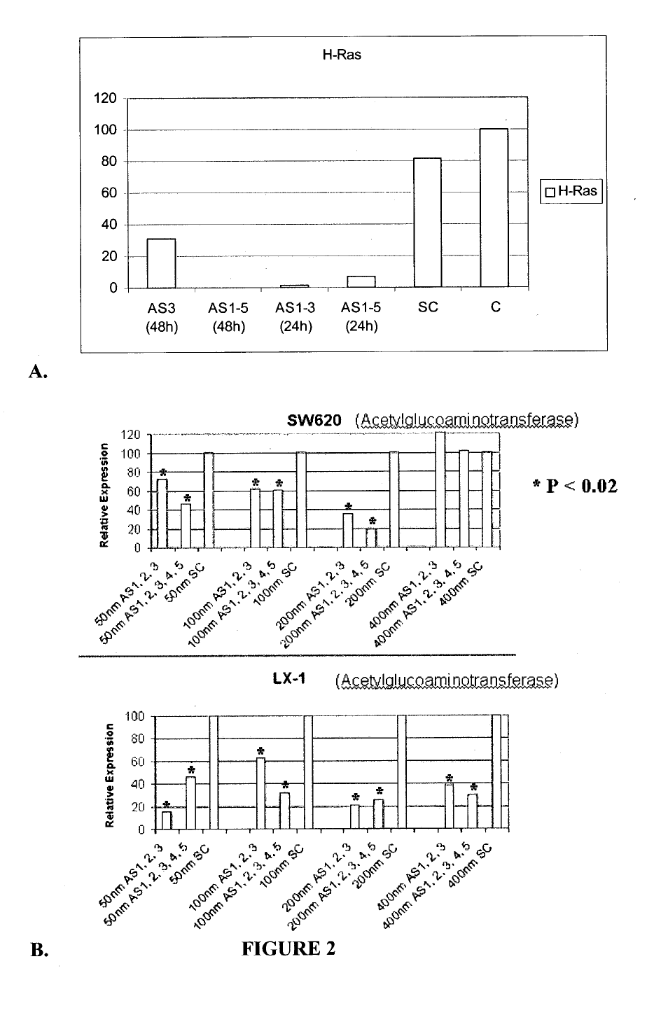

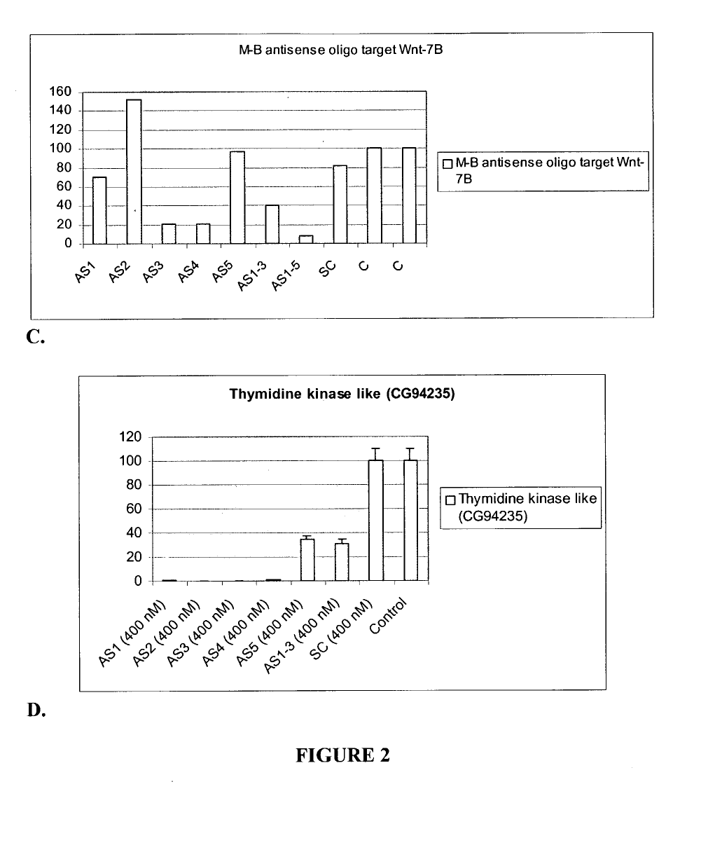

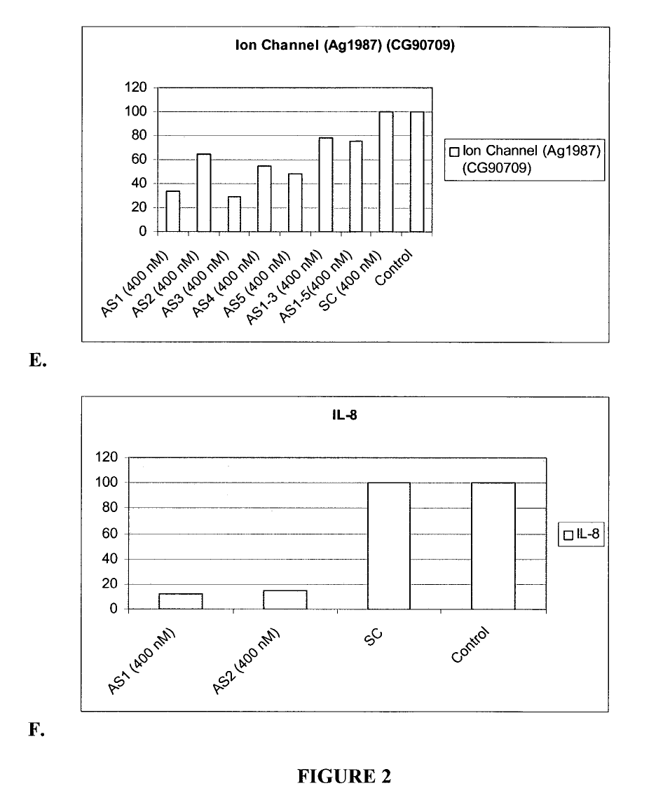

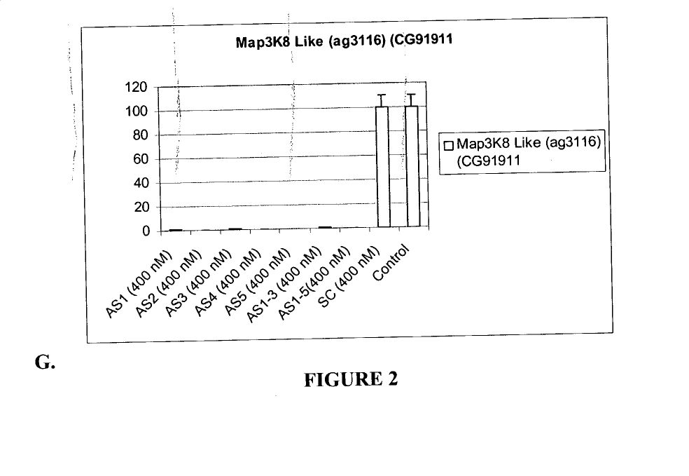

The effects of antisense oligonucleotides (as measured using TaqMan analysis) on the relative expression of different genes are shown in FIG. 2. Expression under various antisense (AS) conditions was compared to scatter control (SC) and control (C) conditions. FIG. 2A shows the relative expression of H-Ras when treated with antisense DNAs. FIG. 2B shows the relative expression of acetylglucoamino-transferase when treated with antisense DNAs in SW60 cells and in LX-1 cells. FIG. 2C shows the relative expression of Wnt-7B when treated with antisense DNAs. FIG. 2D shows the relative expression of the Thymidine kinase like (CG94235) gene when treated with antisense DNAs. FIG. 2E shows the relative expression of the Ion Channel (Ag1987) (CG90709) gene when treated with antisense DNAs. FIG. 2F shows the relative expression of interleukin-8 when treated with antisense DNAs. FIG. 2G shows the relative expression of the Map3K8 like (Ag3116) (CG91911) when treated with antisense DNAs.

Over all, M-B antisense oligos suppressed corresponding target mRNA effectively. For H-Ras (FIG. 2A) and acetylglucoaminotransferase (FIG. 2B), the combination of M-B antisense oligos knocked out mRNA level more effectively than did individual oligos. However, for the thymidine kinase like gene (CG94235) (FIG. 2D), and ion channel gene (CG90709) (FIG. 2E), single M-B antisense oligos worked better than combined oligos. This may result from dilution of an effective oligo by a less effective oligo, or that two or more antisense oligos interact with each other to form a partial hybrid thereby decreasing their effectiveness.

Cell Proliferation Assays

The cell proliferation assay was performed per manufacturer's recommended protocol (Promega, Madison, Wis.).

Transfection Efficiency

To determine the transfection efficiency of both M-B antisense oligo and siRNA, fluorescent labeled M-B antisense oligo (5′-Cy5) and siRNA (5′-FAM) were used to investigate the efficiency of transfection in LX-1, MCF-7, SW620, Hela S3, THP1, MDA-MB-468, and Ramos cells. The labeled cells were analyzed via FACS analysis. In general, the transfection efficiency was high in all tested cell lines ranging from 60 to 97% via FACS analysis.

Comparison of transfection efficiency of a mixed backbone antisense oligonucleotides versus a phosphate-backbone oligonucleotide.

The mixed-backbone oligonucleotide 5′-Cy5CTGAGGCTCTACCGCTGCTT-3′ (SEQ ID NO: XX) was synthesized by The Midland Certified Reagent Company, Inc. (Texas). The concentration was adjusted to 20 uM with sterile DNase-RNase free water and stored in aliquots at −80° C. until used.

Transfection:

Transfection was done in a 24-well plate. Each treatment had triplicate samples. Jurkat cells were prepared on the day of the transfection. Cells were counted and washed with serum-free medium and pellets were re-suspended into serum free RPMI 1640 (Invitrogen, Cat. No 11875). 200 ul of cells (5×10⁵ cells) were plated directly into a 24-well plate.

Transfection reagent mix#1 and mix#2 were made as follows (20 uM oligo stock was used to make a final concentration of 400 nM):

| | Oligonucleotide | Optimem I |
|---|---|---|
| Mix #1: | | |
| 24-well plate | 5 ul | 37.5 ul |
| | Oligofectamine | Optimem I |
| Mix #2: | | |
| 24-well plate | 2 ul | 5.5 ul |

* 5 minutes elapsed before adding mix 2 into mix 1.

After mix #2 was added into mix #1, the combination was mixed gently and incubated at room temperature for 20 minutes. Next, a 50 ul combination of mix 1 and 2 was added into each well containing cells, followed by incubation for 4 hours at 37° C. in a CO₂ incubator. After 4 hour incubation, an additional 250 ul of RPMI 1640 medium containing 30% Fetal Bovine Serum (Invitrogen, Cat No. 10100) was added.

Cells were then incubated at 37° C. in a CO₂ incubator for 18 hours and prepared for FACS analysis by washing with PBS, and resuspending in 1 ml FACS buffer. Analysis was performed by FACSCalibur, gating on live cells, and using FL1 channel for FAM and FL4 channel for Cy5. Plot histograms of each result were superimposed, and presented as cell number vs. relative fluorescence.

Cy5 fluorescence was high in all (100%) of the cells. FAM fluorescence was moderate in approximately half of the cells, and low positive to background in the rest of the cells. Unlabeled oligo gave the background signal level. Jurkat cells are transfected with oligos and Oligofectamine, and mixed backbone oligos were retained at higher levels than phosphate backbone oligos.

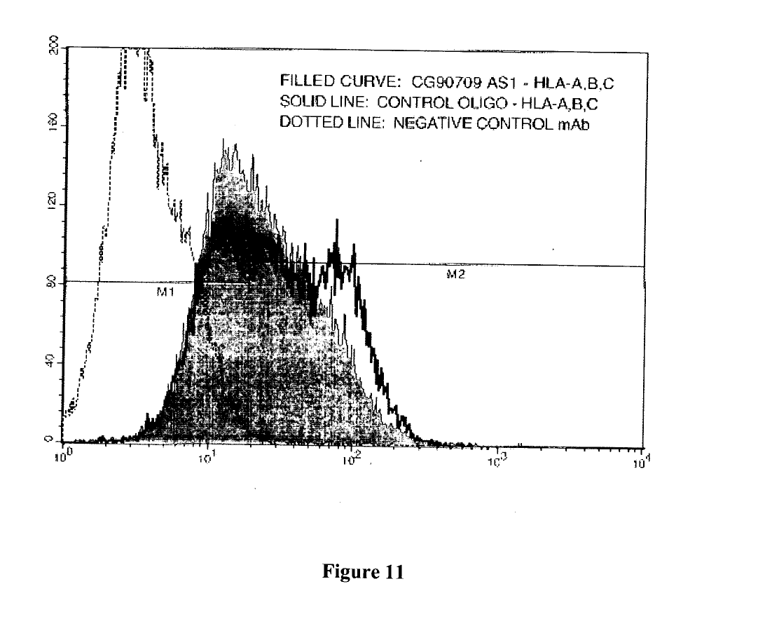

An example of data from FACS (fluorescence activated cell sorting) is shown in FIG. 11. This figure illustrates an analysis of the reduction of the number of cells expressing MHC Class I in response to antisense for novel Ion Channel (CG909709-O₂).

### EXAMPLE 2

### Antisense Inhibition of WNT-7B mRNA Expression

Wnt proteins are secreted ligands that bind to cell surface membrane proteins termed Frizzleds. WNT signaling pathway is implicated in embryogenesis as well as in carcinogenesis. Activation of the Wnt signaling pathway is a major feature of several human neoplasias and appears to lead to the cytosolic stabilization of a transcriptional co-factor, beta-catenin. This co-activator regulates transcription from a number of target genes including oncogenes cyclin D1 and c-myc. There is a correlation between the ability of WNTs to induce beta-catenin accumulation and its transforming potential in vivo. Various wnt genes have been found to be overexpressed in different human cancers, such as breast, gastric and colon cancers, and, accordingly, Wnt antisense oligonucleotides are useful in treating cell proliferative disorders such as breast, gastric and colon cancers.

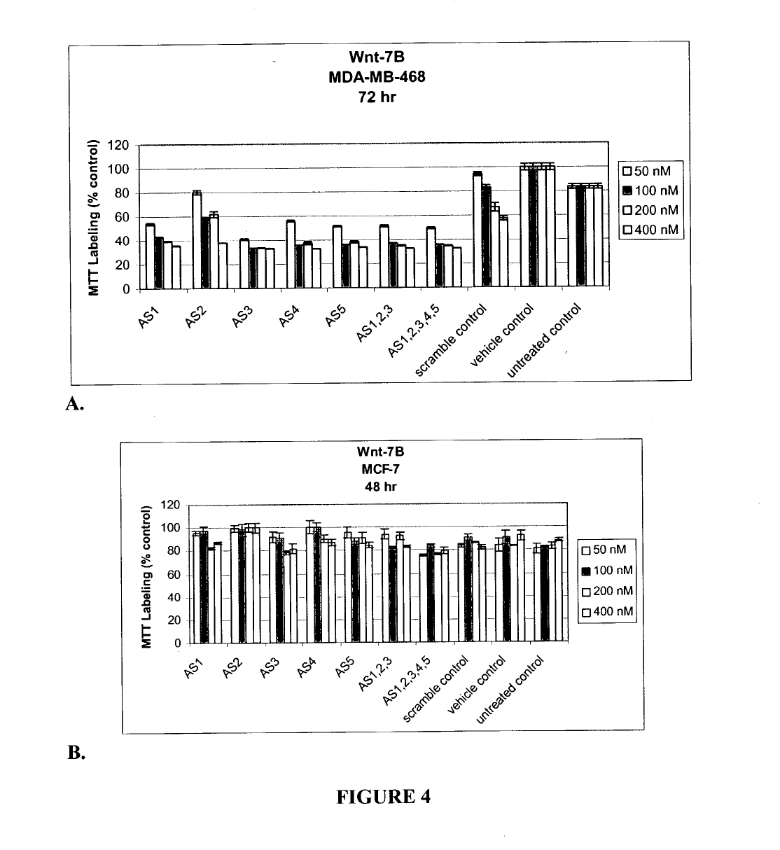

A series of oligonucleotides were designed to target different regions of WNT-7B using the DNA sequence encoding a WNT-7B polypeptide shown in Table 1. Start and stop codons are shown in bold, 5′ and 3′ prime untranslated regions are underlined. The oligonucleotides are shown in Table 2. “Target Site” indicates the first (5′-most) nucleotide number in the particular target sequence to which the oligonucleotide binds. As discussed above, WNT-7B mRNA expression level in the absence or presence of M-B antisense oligonucleotides is shown in FIG. 2C. The effect of various concentrations of Wnt-7B antisense (AS) nucleic acids, scatter control (SC) and control (CTR) on cellular proliferation is shown in FIG. 4. Suppression of MDA-MB-468 cell proliferation in the presence or absence of M-B antisense oligonucleotides is shown in FIG. 4A. FIG. 4B illustrates the effect of various concentrations of Wnt-7B M-B antisense at 72 hours compared to control on cell line MCF-7 (negative control). FIG. 4B shows the change in MCF-7 cell proliferation at various concentrations of Wnt-7B antisense at 48 hours compared to control.

TABLE 1

WNT-7B

(SEQ ID NO:1)

ATGCACAGAAACTTTCGCAAGTGGATTTTCTACGTGTTTCTCTGCTTTGGCGTCCTGTACGTGAAGCTCGGAGC
ACTGTCATCCGTGGTGGCCCTGGGAGCCAACATCATCTGCAACAAGATTCCTGGCCTAGCCCCGCGGCAGCGTG
CCATCTGCCAGAGTCGGCCCGATGCCTCATTGTGATTGGGGGAGGGGGCGCAGATGGGCATCAACGAGTGCCAG
TACCAGTTCCGCTTCGGACGCTGGAACTGCTCTGCCCTCGGCGAGAAGACCGTCTTCGGCAAAGAGCTCCGAGT
AGGGAGCCGTGAGGCTGCCTTCACGTACGCCATCACCGCGGCTGGCGTGGCGCACGCCGTCACCGCTGCCTGCA
GCCAAGGGAACCTGAGCAACTGCGGCTGCGACCGCGAGAAGCAGGGCTACTACAACCAAGCCGAGGGCTGGAAG
TGGGGCGGCTGCTCGGCCGACGTGCGTTACGGACTCGACTTCTCCCGGCGCTTCGTGGACGCTCGGGAGATCAA
GAAGAACGCGCGGCGCCTCATGAACCTGCATAACAATGAGGCCGGAAGGAAGGTTCTAGAGGACCGGATGCAGC
TGGAGTGCAAGTGCCACGGCCGTGTCTGGCTCCTGCACCACAAAACCTGCTGGACCACGCTGCCCAAGTTCCGA
GAGGTGGGCCACCTGCTGAAGGAGAAGTACCACGCGGCCGTGAAGGTGGAGGTGGTGCGGGCCAGCCGTCTGCG
GCAGCCCACCTTCCTGCGCATCAAACAGCTGCGCAGCTATCAGAAGCCCATGGAGACAGACCTGGTGTACATTG
AGAAGTCGCCCACTACTGCGAGGAGGACGCGGCAACGGGAAGCGTGGGCACGCAGCGCCGGTCTCTGCAACCGC
ACGTCGCCCGGCGCGGACGACTGTGACACCATGTGCTGCGGCCGAGGCTACAACACCCACCAGTACACCAAGGT
GTGGCAGTGCAACTGCAAATTCCACTGGTGCTGCTTCGTCAAGTGCAACACCTGCAGCGAGCGCACCGAGGTCT
TCACCTGCAAGTGAGCCAGGCCCGGAGGCGGCCC

TABLE 2

Oligonucleotides

| | | | |
|---|---|---|---|
| | | Target | SEQ ID |
| Curagen # | Sequence | Site | NO |

| | | | |
|---|---|---|---|
| CG51932-01-AS1 | TTGCGAAAGTTTCTGTGCAT | 1 | 7 |
| CG51932-01-AS2 | TACAGGACGCCAAAGCAGAG | 40 | 8 |
| CG51932-01-AS3 | ACAGTGCTCCCAGCTTCACG | 60 | 9 |
| CG51932-01-AS4 | GTCGATGCCGTAACGCACGT | 464 | 10 |
| CG51932-01-AS5 | CTTGCAGGTGAAGACCTCGG | 1028 | 11 |

### EXAMPLE 3

### Antisense Inhibition of N-acetylglucosaminyltransferase mRNA Expression

N-acetylglucosaminyltransferases catalyze the addition of the bisecting GIcNAc to the core of N-glycans. These proteins have been associated with tumor progression, cell migration and matrix invasion, tumor metastasis, enhanced cell survival, some downstream of ras and PDGF signaling pathways. N-acetylglucosaminyltransferases increase the prevalence of mammary tumors. Thus, antisense oligonucleotides for acetylglucosaminyltransferases are useful in treating cell proliferative disorders.

A series of oligonucleotides were designed to target different regions of N-acetylglucosaminyltransferase using the DNA sequence encoding an N-acetyl-glucosaminyltransferase polypeptide shown in Table 3. The oligonucleotides are shown in Table 4. “Target Site” indicates the first (5′-most) nucleotide number in the particular target sequence to which the oligonucleotide binds.

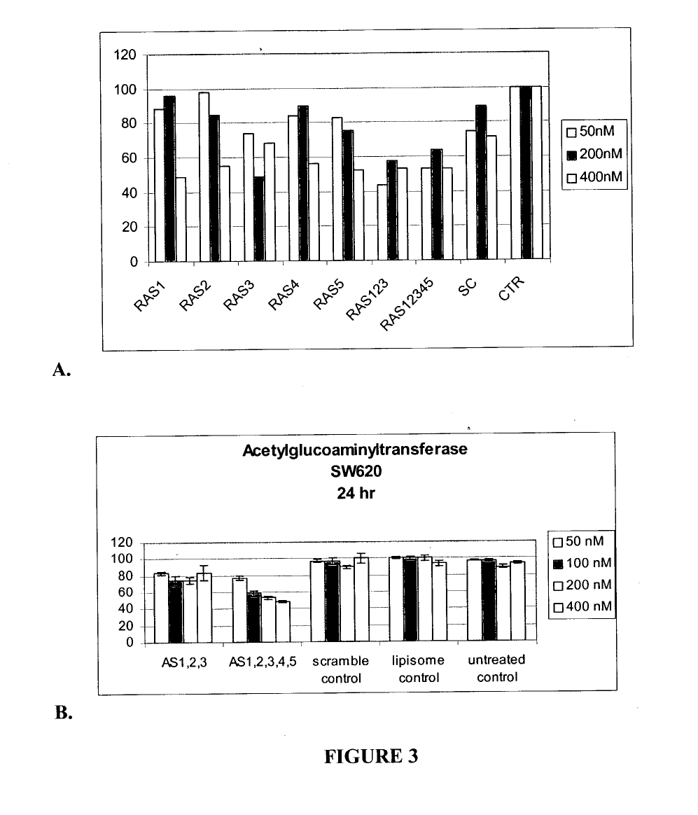

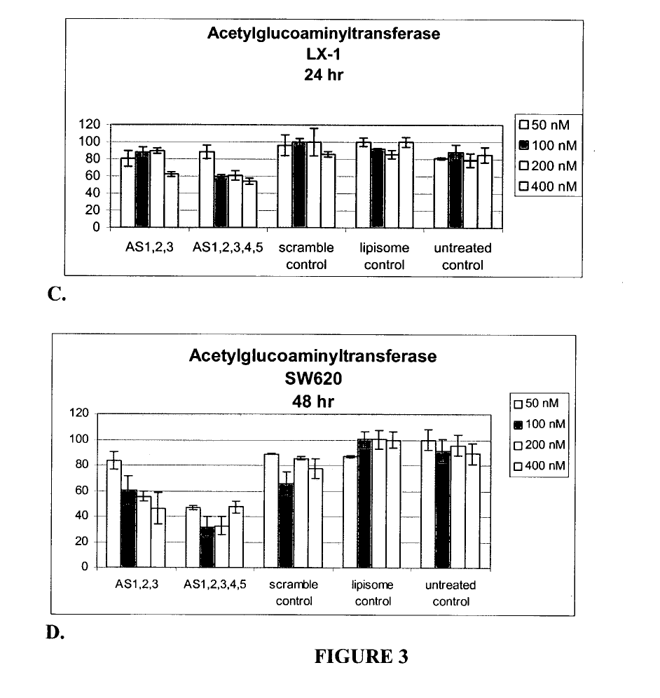

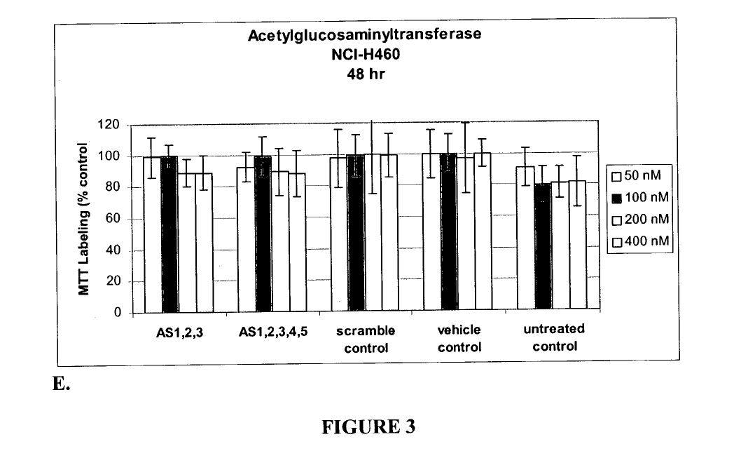

As discussed above, N-acetylglucosaminyltransferase mRNA expression level in the absence or presence of M-B antisense oligonucleotides is shown in FIG. 2B. The effect of various concentrations of N-acetylglucosaminyltransferase antisense (AS) nucleic acids, scatter control (SC) and control (CTR) on cellular proliferationis is shown in FIG. 3. Suppression of SW620 cell proliferation at various concentrations of acetylglucoaminyltransferase antisense compared to control at 24 hours is shown in FIG. 3B. FIG. 3C shows the change in LX-1 cell proliferation at various concentrations of acetylglucoaminyltransferase antisense compared to control at 24 hours. FIG. 3D shows the change in SW620 cell proliferation at various concentrations of acetylglucoaminyltransferase antisense compared to control at 48 hours. FIG. 3E shows the change in NCI-H460 cell proliferation (negative control)at various concentrations of acetylglucoaminyltransferase antisense compared to control at 48 hours.

TABLE 3

N-acetyl-glucosaminyltransferase

(SEQ ID NO:2)

TAAAAATACAAAAAATTAGCCGGGCGTAGTGGCGGGCGCCTGTAGTCCCAGCTACTTGGGAGGCTGAGGCAGGA
GAATGGCCGTGACCCGGGAGGCACAGCTTGCAGTGAGCCGAGATCCCGCCACTGCACTCCAGCCTGGGCGACAG
AGCGAGACTCCGTCTCAAAAAAAAAAAAAAAGAACATCCTGAGCCGGGCGTGGAAAAGCTCTTTGCAGATGGCG
CTTCCATCTCTGCGCCCCTCGGGGTGGGGGCTGTCCAATGTTGCTCCTGCTGGGCCCTCTCAGGCTTCCTCTTT
GCCCACCCAAAAGGAAAATCCACTGCACCTCCACTTGGTGACTGACGCCGTGGCCAGAAACATCCTGGAAGACG
CTCTTCCACACATGGATGGTGCCTGCTGTCCGTGTCAGCTTTTATCATGCCGACCAGCTCAAGCCCCAGGTCTC
CTGGATCCCCAACAAGCACTACTCCGGCCTCTATGGGCTCATGCAGCTGGTGCTGCCAAGTGCCTTGCCTGCTG
AGCTGGCCCGCGTCATTGTCCTGGACACGGATGTCACCTTCGCCTCTGACATCTCGGAGCTCTGGGCCCTCTTT
GCTCACTTTTCTGACACGCAGGCGATCGGTCTTGTGGAGCACAAGAGTGACTGGTACCTGGGCACCCTCTGGAA
GACCACAGGCCCTGGCCTGCCTTGGGCCGGGGATTTAACACCAGGTGTGATCCTGCTGCGGCTGCACCGGCTCC
GGCAGGCTGGCTGGGAGCAGATGTGGAGGCTGACAGCAAGGCGGGAGCTCCTTAGCCTGCCTGCCACCTCACTG
GCTGACCAGGACATCTTCACGCTGTGATCCAGGAGCACCCGGGGCTAGTGCAGCGTCTGCCTTGTGTCCTGGAA
TGTGCAGCTGTCGATCACACACTGGCCCGAGCGCTGCTACTCTGAGGCGTCTGACCTCAAGGTGATCAACTGGA
ACTCACCAAAGAAGCTTCGGGTGAAGAACAAGCATGTGGAATTCTTCCGCCATTTCTACCTGACCTTCCTGGAG
TACGATGGGAACCTGCTGCGGAGAGAGCTCTTTGTGTGCCCCAGCCAGCCCCAACCTGGTGCTGAGCAGTTGTA
GCAGGCCCTGGCACACTGGACGAGGAAGACCCCTGCTTTGAGTTCCGGCAGCAGCAGCTAACTGTGCACCGTG
TGCATGTCCTTTCCTGCCCCATGAACCGCCACCCCCCCGGCCTCACGATGTAACCCTTGTGGCCCAGCTCTCC
ATGGACCGGCTGCAGATGTTGGAAGCCCTGTGCAGGCACTGGCCTGGCCCAATGAGCCTGGCCTTGTACCTGAC
AGACGCA

TABLE 4

Oligonucleotides

| | | | |
|---|---|---|---|
| | | Target | SEQ ID |
| Curagen # | Sequence | Site | NO |

| | | | |
|---|---|---|---|
| CG51475-01-AS1 | CAGCAGGAGCAACATGGGAC | 255 | 12 |
| C051475-01-AS2 | CAAAGAGGAAGCCTGAGAGG | 278 | 13 |
| CG51475-01-AS3 | CCAAGTGGAGGTGCAGTGGA | 316 | 14 |
| CG51475-01-AS4 | GAGGTGGCAGGCAGGCTAAG | 791 | 15 |
| CG51475-01-AS5 | CTACAACTGCTCAGCACCAG | 1092 | 16 |

### EXAMPLE 4

### Antisense Inhibition of Voltage-gated K Channel mRNA Expression

Potassium channels represent a complex class of voltage-gated ion channels. These channels maintain membrane potential, regulate cell volume, and modulate electrical excitability in neurons. The delayed rectifier function of potassium channels allows nerve cells to efficiently repolarize following an action potential. Voltage gated potassium channel oligonucleotides are useful in the treatment of neurological disorders such as epilepsy, and cardiac disorders involving arrhythmias.

A series of oligonucleotides were designed to target different regions of the Voltage-gated K channel using the DNA sequence encoding a Voltage-gated K channel polypeptide shown in Table 5. The oligonucleotides are shown in Table 6. “Target Site” indicates the first (5′-most) nucleotide number in the particular target sequence to which the oligonucleotide binds.

TABLE 5

Voltage Gated K channel

(SEQ ID NO:3)

GTCTGAGTCACAGAGATGGGCAAGATCGAGAACAACGAGAGGGTGATCCTCAATGTCGGGGGCACCCGGCACGA
AACCTACCGCAGCACCCTCAAGACCCTGCCTGGAACACGCCTGGCCCTTCTTGCCTCCTCCGAGCCCCCAGGCG
ACTGCTTGACCACGGCGGGCGACAAGCTGCAGCCGTCGCCGCCTCCACTGTCGCCGCCGCCGAGAGCGCCCCCG
CTGTCCCCCGGGCCAGGCGGCTGCTTCGAGGGCGGCGCGGGCAACTGCAGTTCCCGCGGCGGCAGGGCCAGCGA
CCATCCCGGTGGCGGCCGCGAGTTCTTCTTCGACCGGCACCCGGGCGTCTTCGCCTATGTGCTCAATTACTACC
GCACCGGCAAGCTGCACTGCCCCGCAGACGTGTGCGGGCCGCTCTTCGAGGAGGAGCTGGCCTTCTGGGGCATC
GACGAGACCGACGTGGAGCCCTGCTGCTGGATGACCTACCGGCAGCACCGCGACGCCGAGGAGGCGCTGGACAT
CTTCCAGACCCCCGACCTCATTGGCGGCGACCCCGGCGACGACGAGGACCTGGCGGCCAAGAGGCTGGGCATCG
AGGACGCGGCGGGGCTCGGCGGCCCGGACGGCAAATCTGGCCGCTGGAGGAGGCTGCAGCCCCGCATGTGGGCC
CTCTTCGAAGACCCCTACTCGTCCACAGCCGCCAGGTTTATTGCTTTTGCTTCTTTATTCTTCATCCTGGTTTC
AATTACAACTTTTTGCCTGGAAACACATGAAGCTTTCAATATTGTTAAAAACAAGACAGAACCAGTCATCAATG
GCACAAGTGTTGTTCTACAGTATGAAATTGAAACGGATCCTGCCTTGACGTATGTAGAAGGAGTGTGTGTGGTC
TGGTTTACTTTTGAATTTTTAGTCCGTATTGTTTTTTCACCCAATAAACTTGAATTCATCAAAAATCTCTTCAA
TATCATTGACTTTGTGGCCATCCTACCTTTCTACTTAGAGGTGGGACTCAGTGGGCTGTCATCCAAAGCTCCTA
AAGATGTGCTTGGCTTCCTCAGGGTGGTAAGGTTTGTGAGGATCCTGAGAATTTTCAAGCTCACCCGCCATTTT
GTAGGTCTGAGGGTGCTTGGACATACTCTTCGAGCTAGTACTAATGAATTTTTGCTGCTGATAATTTTCCTGGC
TCTAGGAGTTTTGATATTTGCTACCATGATCTACTATGCCGAGAGAGTGGGAGCTCAACCTAACGACCCTTCAG
CTAGTGAGCACACACAGTTCAAAAACATTCCCATTGGGTTCTGGTGGGCTGTAGTGACCATGACTACCCTGGGT
TATGGGGATATGTACCCCCAAACATGGTCAGGCATGCTGGTCGGAGCCCTGTGTGCTCTGGCTGGAGTGCTGAC
AATAGCCATGCCAGTGCCTGTCATTGTCAATAATTTTGGAATGTACTACTCCTTGGCAATGGCAAAGCAGAAAC
TTCCAAGGAAAAGAAAGAAGCACATCCCTCCTGCTCCTCAGGCAAGCTCACCTACTTTTTGCAAGACAGAATTA
AATATGGCCTGCAATAGTACACAGAGTGACACATGTCTGGGCAAAGACAATCGACTTCTGGAACATAACAGATC
AGTGTTATCAGGTGACGACAGTACAGGAACTGAGCCGCCACTATCACCCCCAGAAAGGCTCCCCATCAGACGCT
CTAGTACCAGAGACAAAAACAGAAGAGGGGAAACATGTTTCCTACTGACGACAGGTGATTACACGTGTCCTTCT
GATGGAGGGATCAGGAAAGGTTATGAAAAATCCCGAAGCTTAAACAACATAGCGGGCTTGGCAGGCAATGCTCT
GAGGCTCTCTCCAGTAACATCACCCTACAACTCTCCTTGTCCTCTGAGGCGCTCTCGATCTCCCATCCCATCTA
TCTTGTAAACCAAACAACCAAACTGCATC

TABLE 6

Oligonucleotides

| | | | |
|---|---|---|---|
| | | Target | SEQ ID |
| Curagen # | Sequence | Site | NO |

| | | | |
|---|---|---|---|
| CG50249-01-AS1 | GTTCTCGATCTTGCCCATCT | 14 | 17 |
| CG50249-01-AS2 | ATTGAGGATCACCCTCTCGT | 35 | 18 |
| CG50249-01-AS3 | GGTGCTGCGGTAGGTTTCGT | 71 | 19 |
| CG50249-01-AS4 | CTGTGTGTGCTCACTAGCTG | 1256 | 20 |
| CG50249-01-AS5 | GTTTACAAGATAGATGGGAT | 1915 | 21 |

The Scramble Control oligo was: 5′-CTGAGGCTCTACCGCTGCTT-3′ (SEQ ID NO:22).

### EXAMPLE 5

### Antisense Inhibition of Ion Transport mRNA Expression

A series of oligonucleotides were designed to target different regions of an Ion Transport channel using the DNA sequence encoding an Ion Transport channel polypeptide shown in Table 7. The oligonucleotides are shown in Table 8. “Target Site” indicates the first (5′-most) nucleotide number in the particular target sequence to which the oligonucleotide binds.

TABLE 7

Ion Transport Channel (Ag 1987)

(SEQ ID NO:4)

TTAATCTTCTGTCGCAGAAATGCAATGGCACATCGTGATTCTGAGATGAAAGAAGAATGTCTAAGGGAAGACCT
GAAGTTTTACTTCATGAGCCCTTGTGAAAAATACCGAGCAGACGCCACAATTCCGTGGAAACTGGGTTTGCAGA
TTTTGAAGATAGTCATGGTCACCACACAGCTTGTTCGTTTTGGTTTAAGTAACCAGCTGGTGGTTGCTTTCAAA
GAAGATAACACTGTTGCTTTTAAGCACTTGTTTTTGAAAGGATATTCTGGTACAGATGAAGATGACTACAGCTG
CAGTGTATATACTCAAGAGGATGCCTATGAGAGCATCTTTTTTCCTATTAATCAGTATCATCAGCTAAAGGACA
TTACCCTGGGCACCCTTGCTTATGGAGAAAATGAAGACAATAGAATTGGCTTAAAAGTCTGTAAGCAGCATTAC
AAGAAAGGGACCATGTTTCCTTCTAATGAGACACTGAATATTGACAACGACGTTGAGCTCAACTGTGGGGTTCT
GGCGATATACATTTTAAAGTGTTATTCCCTAAGAGATATTATGACAATTTATACCTTTCAATATATTTTATTCA
GGCTCTTACAGGTTGAAATCTCCTTTCATCTTAAAGGCATTGACCTACAGACAATTCATTCCCGTGAGTTACCA
GACTGTTATGTCTTTCAGAATACGATTATCTTTGACAATAAAGCTCACAGTGGCAAAATCAAAATCTATTTTGA
CAGTGATGCCAAAATTCAAGAATGTAAAGACTTGAACATATTTGGATCTAGTAAGTATGCTCTGGTGTTTGATG
CATTTGTCATTGTGATTTGCTTGGCATCTCTTATTCTGTGTACAAGATCCATTGTTCTTGCTCTAAGGTTACGG
AGATTTCTAAATTTCTTCCTGGAGAAGTACAAGCGGCCTGTGTGTGACACCGACCAGTGGGAGTTCATCAACGG
CTGGTATGTCCTGGTGATTATCAGCGACCTAATGACAATCATTGGCTCCATATTAAAAATGGAAATCAAAGCAA
AGAATCTCACAAACTATGATCTCTGCAGCATTTTTCTTGGAACCTCTACGCTCTTGCTTTGGGTTGGAGTCATC
AGATACCTGGGTTATTTCCAGGCATATAATGTACTGATTTTAACAATGCAGGCCTCACTGCCAAAAGTTCTTCG
GTTTTGTGCTTGTGCTGCTATGATTTATCTGGGTTACACATTCTGTGGCTGGATTGTCTTAGGACCATACCATC
TACAGTTTGAAAATCTGAACACAGTTGCTGAGTGTCTGTTTTCTCTGGTCAACGGTGATGACATGTTTGCAACC
TTTGCCCAAATCCAGCAGAAGAGCATCTTGGTGTGGCTGTTCAGTCGTCTGTATTTATATTCCTTCATCAGCCT
TTTTATATATATGATTCTCAGTCTTTTTATTGCACTTATTACAGATTCTTATGACACCATTAAGAAATTCCAAC
AGAATGGGTTTCCTGAAACGGATTTGCAGGAATTCCTGAAGGAATGCAGTAGCAAAGAAGAGTATCAGAAAGAG
TCCTCAGCCTTCCTGTCCTGCATCTGCTGTCGGAGGAGGTCAGTATCATGTTTATTCTCCATGCTCCTGAGATG
GGCTGTTCTGTTGTCTTAAGAAAGAGCCCCTCCAAGATTACCATTACAT

TABLE 8

Oligonucleotides

| | | | |
|---|---|---|---|
| | | Target | SEQ ID |
| Curagen # | Sequence | Site | NO |

| | | | |
|---|---|---|---|
| CG90709-01-AS1 | GAATCACGATGTGCCATTGC | 22 | 23 |
| CG90709-01-AS2 | GACATTCTTCTTTCATCTCA | 42 | 24 |
| CG90709-01-AS3 | GAATCTGGCGTCTGGCTCGG | 108 | 25 |
| CG90709-01-AS4 | CTCCCACTGGTCGGTGTCAC | 932 | 26 |
| CG90709-01-AS5 | CCTCCGACAGCAGATGCAGG | 1571 | 27 |

As discussed above, Ion Transport mRNA expression level in the absence or presence of M-B antisense oligonucleotides is shown in FIG. 2E. The FACS results for the analysis of reduction of number of cells expressing MHC class I in response to antisense for the ion channel mRNA.

### EXAMPLE 6

### Antisense Inhibition of Map3K8 mRNA Expression

The MAPK cascades regulate a wide variety of cellular functions, including cell proliferation, differentiation, and stress responses. Mitogen-activated protein kinase kinase kinase 8 (MAP3K8) is associated with cell proliferation and cancer, accordingly antisense MAP3K8 oligonucleotides are useful in treating cell proliferative disorders such as cancer.

A series of oligonucleotides were designed to target different regions of Map3K8 using the DNA sequence encoding a Map3K8 polypeptide shown in Table 9. The oligonucleotides are shown in Table 10. “Target Site” indicates the first (5′-most) nucleotide number in the particular target sequence to which the oligonucleotide binds. As discussed above, Map3K8 mRNA expression level in the absence or presence of M-B antisense oligonucleotide is shown in FIG. 2G.

TABLE 9

Map3K8

(SEQ ID NO:5)

TATGTCAGTTTCCCATGGGTCTTGAATGCAAATACAAATATCGTAAACTAAATATTTGTGTTTTCTTTCCTAGA
CTCTCCAGAAAGAGCAACAGTAATGGAGTACATGAGCACTGGAAGTGACAATAAAGAAGAGATTGATTTATTAA
TTAAACATTTAAATGTGTCTGATGTAATAGACATTATGGAAAATCTTTATGCAAGTGAAGAGCCAGCAGTTTAT
GAACCCAGTCTAATGACCATGTGTCAAGACAGTAATCAAAACGATGAGCGTTCTAAGTCTCTGCTGCTTAGTGG
CCAAAAGGTACCATGGTTGTCATCAGTCAAATACGGAACTGTGGAGGATTTGCTTGCTTTTGAAAACCATATAT
CCAACACTGCAAAGCATTTTTATGTTCAACGACCACAGGAATATGGTATTTTATTAAACATGGTAATCACTCCC
CAAAATGGACGTTACCAAATAGATTCCGATGTTCTCCTGATCCCCTGGAAGCTGACTTACAGGAATATTGGTTC
TGATTTTATTCCTCGGGGCGCCTTTGGAAAGGTATACTTGGCACAAGATATAAAGACGAAGAAAAGAATGGCGT
GTAAACTGATCCCAGTAGATCAATTTAAGCCATCTGATGTGGAAATCCAGGCTTGCTTCCGGCACGAGAACATC
GCAGAGCTGTATGGCGCAGTCCTGTGGGGTGAAACTGTCCATCTCTTTATGGAAGCAGGCGAGGGAGGGTCTGT
TCTGGAGAAACTGGAGAGCTGTGGACCAATGAGAGAATTTGAAATTATTTGGGTGACAAAGCATGTTCTCAAGG
GACTTGATTTTCTACACTCAAAGAAAGTGATCCATCATGATATTAAACCTAGCAACATTGTTTTCATGTCCACA
AAAGCTGTTTTGGTGGATTTTGGCCTAAGTGTTCAAATGACCGAAGATGTCTATTTTCCTAAGGACCTCCGAGG
AACAGAGATTTACATGAGCCCAGAGGTCATCCTGTGCAGGGGCCATTCAACCAAAGCAGACATCTACAGCCTGG
GGGCCACGCTCATCCACATGCAGACGGGCACCCCACCCTGGGTGAAGCGCTACCCTCGCTCAGCCTATCCCTCC
TACCTGTACATAATCCACAAGCAAGCACCTCCACTGGAAGACATTGCAGATGACTGCAGTCCAGGGATGAGAGA
GCTGATAGAAGCTTCCCTGGAGAGAAACCCCAATCACCCCCCAAGAGCCGCAGACCTACTAAAACATGAGGCCC
TGAACCCGCCCACAGAGGATCAGCCACGCTGTCAGAGTCTGGACTCTGCCCTCTTGGAGCGCAAGAGCCTGCTG
AGTAGGAAGGAGCTCGAACTTCCTGAGAACATTGCTGATTCTTCGTGCACAGGAAGCACCGAGGAATCTGAGAT
GCTCAAGAGGCAACGCTCTCTCTACATCGACCTCGGCGCTCTGGCTGGCTACTTCAATCTTGTTCGGGGACCAC
CAACGCTTGAATATCGCTGAAGGATGCCATGTTTGCTCTAAATTAAGACAGCATTGATCTCCTGGAGGCTGGTT
CTGCTGCCTCTACACAGGGGCCCTGTACAGTGAATGGTGCCATTTTCGAAGGAGCAGTGTGACCTCCTGTGACC
CGTGAATGTGCCTCCAAGCGGCCCTGTGTGTTTGACATGTGAAGCTATTTGATATGCACCAGGTCTCAAGGTTC
TCATTTCTCAGGTCACGTGATTCTAAGGCAGGAATTTGAGAGTTCACAGAAGGATCGTGTCTGCTGACTGTTTC
ATTCACTGTGCACTTTGCTCAAAATTTTAAAAATACCAATCACAAGGATAATAGAGTAGCCTAAAATTACTATT
CTTGGTTCTTATTTAAGTATGGAATATTCATTTTACTCAGAATAGCTGTTTTGTGTATATTGGTGTATATTATA
TAACTCTTTGAGCCTTTATTGGTAAATTCTGGTATACATTGAATTCATTATAATTTGGGTGACTAGAACAACTT
GAAGATTGTAGCAATAAGCTGGACTAGTGTCCTAAAAATGGCTAACTGATGAATTAGAAGCCATCTGACAGCAG
GCCACTAGTGACAGTTTCTTTTGTGTTCCTATGGAAACATTTTATACTGTACATGCTATGCTGAAGACATTCAA
AACGTGATGTTTTGAATGTGGATAAAACTGTGTAAACCACATAATTTTTGTACATCCCAAAGGATGAGAATGTG
ACCTTTAAGAAAAATGAAAACTTTTGTAAATTATTGATGATTTTGTAATTCTTATGACTAAATTTTCTTTTAAG
CATTTGTATATTAAAATAGCATACTGTGTATGTTTTATATCAAATGCCTTCATGAATCTTTCATACATATATAT
ATTTGTAACATTGTAAAGTATGTGAGTAGTCTTATGTAAAGTATGTTTTTACATTATGCAAATAAAACCCAATA
CTTTTGTCCAATGTGGTTGGTCAAATCAACTGAATAAATTCAGTATTTTCCCTT

TABLE 10

Oligonucleotides

| | | | |
|---|---|---|---|
| | | Target | SEQ ID |
| Curagen # | Sequence | Site | NO |

| | | | |
|---|---|---|---|
| CG91911-01-AS1 | TGCTCATGTACTCCATTACT | 93 | 28 |
| CG91911-01-AS2 | TTCTTTATTGTCACTTCCAG | 113 | 29 |
| CG91911-01-AS3 | TGCTGGCTCTTCACTTGCAT | 197 | 30 |
| CG91911-01-AS4 | CATGTGGATGAGCGTGCCCC | 1037 | 31 |
| CG91911-01-AS5 | CCATATTCAAGCGTTGGTGG | 1477 | 32 |

### EXAMPLE 7

### Effect of Thymidine Kinase Antisense on Il-1b Secretion

Thymidylate kinase catalyzes the phosphorylation of dTMP to form dTDP in the dTTP synthesis pathway for DNA synthesis. Antisense Thymidine kinase oligonucleotides are useful in treating cell proliferative disorders and modulating the expression of II-1b.

A series of oligonucleotides were designed to target different regions of Thymidine kinase using the DNA sequence encoding a Thymidine kinase polypeptide shown in Table 11. The oligonucleotides are shown in Table 12. “Target Site” indicates the first (5′-most) nucleotide number in the particular target sequence to which the oligonucleotide binds. As discussed above, Thymidine kinase mRNA expression level in the absence or presence of M-B antisense oligonucleotide is shown in FIG. 2D.

TABLE 11

Thymidine Kinase

(SEQ ID NO:6)

GGGCGGCGCGGGGTCTGCGCTGGGGCCATGGCTCCGCCGCGCCGCTTCGTCCTGGAGCTTCCCGACTGCACCCT
GGCTCACTTCGCCCTAGGCGCCGACGCCCCCGGCGACGCAGACGCCCCCGACCCCCGCCTGGCGGCGCTGCTGG
GGCCCCCGGAGCGCAGCTACTCGCTGTGCGTGCCCGTGACCCCGGACGCCGGCTGCGGGGCCCGGGTCCGGGCG
GCGCGGCTGCACCAGCGCCTGCTGCACCAGCTGCGCCGCGGCCCCTTCCAGCGGTGCCAGCTGCTCAGGCTGCT
CTGCTACTGCCCGGGCGGCCAGGCCGGCGGCGCACAGCAAGGCTTCCTGCTGCGCGACCCCCTGGATGACCCTG
ACACCCGGCAAGCGCTGCTCGAGCTGCTGGGCGCCTGTCAGGAGGCACCACGCCCGCACTTGGGCGAGTTCCAG
GCCGACCCGCGCGGCCAGCTGTGGCAGCGCCTCTGGGAGGTGCAAGACGGCAGGCGGCTGCAGGTGGGCTGCGC
ACAGGTCGTGCCCGTCCCGGAGCCCCCGCTGCACCCGGTGGTGCCAGACTTGCCCAGTTCCGTGGTCTTCCCGG
ACCGGGAAGCCGCCCGGGCCGTTTTGGAGGAGTGTACCTCCTTTATTCCTGAAGCCCGGGCAGTGCTTGACCTG
GTCGACCAGTGCCCAAAACAGATCCAGAAAGGAAAGTTCCAGGTTGTTGCCATCGAAGGACTGGATGCCACGGG
TGGTAAAACCACGGTGACCCAGTCAGTGGCAGATTCACTTAAGGCTGTCCTCTTAAAGTCACCACCCTCTTGCA
TTGGCCAGTGGAGGAAGATCTTTGATGATGAACCAACTATCATTAGAAGAGCTTTTTACTCTTTGGGCAATTAT
ATTGTGGCCTCCGAAATAGCTAAAGAATCTGCCAAATCTCCTGTGATTGTAGACAGGCACAGCACGGCCACCTA
TGCCATAGCCACTGAGGTGAGTGGGGGTCTCCAGCACCTGCCCCCAGCCCATCACCCTGTGTACCAGTGGCCAG
AGGACCTGCTCAAACCTGACCTTATCCTGCTGCTCACTGTGAGTCCTGAGGAGAGGTTGCAGAGGCTGCAGGGC
CGGGGCATGGAGAAGACCAGGGAAGAAGCAGAACTTGAGGCCAACAGTGTGTTTCGTCAAAAGGTAGAAATGTC
CTACCAGCGGATGGAGAATCCTGGCTGCCATGTGGTTGATGCCAGCCCCTCCAGAGAAAAGGTCCTGCAGACGG
TATTAAGCCTAATCCAGAATAGTTTTAGTGAACCGTAGTTACTCTGGCCAGGTGCCACGTCTAACTAGATTAGA
TGTTGTTTGAAACATCTACATCCACCATTTGTTATGCAGTGTTCCCAAATTTCTGTTCTACAAGCATGTTGTGT
GGCAGAAAACTGGAGACCAGGCATCTTAATTTTACTTCAGCCATCGTACCCTCTTCTGACTGATGGACCCGTCA
TCACAAAGGTCCCTCTCATCATGTTCCAGTGAGAGGCCAGCGATTGCTTTCTTCCTGGCATAGTAAACATTTTC
TTGGAACATATGTTTCACTTAATCACTACCAAATATCTGGAAGACCTGTCTTACTCAGACAGCACCAGGTGTAC
AGAAGCAGCAGACAAGATCTTCCAGATCAGCAGGGAGACCCCGGAGCCTCTGCTTCTCCTACACTGGCATGCTG
ATGAGATCGTGACATGCCCACATTGGCTTCTTCCACATCTGGTTGCACTCGTCATGATGGGCTCGCTGCATCTC
CCTCAGTCCCAAATTCTAGAGCCAAGTGTTCCTGCAGAGGCTGTCTATGTGTCCTGGCTGCCCAAGGACACTCC
TGCAGAGCCATTTTTGGGTAAGGAACACTTACAAAGAAGGCATTGATCTTGTGTCTGAGGCTCAGAGCCCTTTT
GATAGGCTTCTGAGTCATATATAAAGACATTCAAGCCAAGATGCTCCAACTGCAAATATACCAACCTTCTCTGA
ATTATATTTTGCTTATTTATATTTCTTTTCTTTTTTTCTAAAGTATGGCTCTGAATAGAATGCACATTTTCCAT
TGAACTGGATGCATTTCATTTAGCCAATCCAGTAATTTATTTATATTAATCTATACATAATATGTTTCCTCAGC
ATAGGAGCTATGATTCATTAATTAAAAGTGGAGTCAAAACGCTAAATGCAATGTTTGTTGTGTATTTTCATTAC
ACAAACTTAATTTGTCTTGTTAAATAAGTACAGTGGATCTTGGAGTGGGATTTCTTGGTAAATTATCTTGCACT
TGAATGTCTCATGATTACATATGAAATCGCTTTGACATATCTTTAGACAGAAAAAAGTAGCTGAGTGAGGGGGA
AATTATAGAGCTGTGTGACTTTAGGGAGTAGGTTGAACCAGGTGATTACCTAAAATTCCTTCCAGTTCAAAGGC
AGATAAATCTGTAAATTATTTTATCCTATCTACCATTTCTTAAGAAGACATTACTCCAAAATAATTAAATTTAA
GGCTTTATCAGGTCTGCATATAGAATCTTAAATTCTAATAAAGTTTCATGTTAATGTCATAGGATTTTTAAAAG
AGCTATAGGTAATTTCTATATAATATGTGTATATTAAAATGTAATTGATTTCAGTTGAAAGTATTTTAAAGCTG
ATAAATAGCATTAGGGTTCTTTGCAATGTGGTATCTAGCTGTATTATTGGTTTTATTTACTTTAAACATTTTGA
AAAGCTTATACTGGCAGCCTAGAAAAACAAACAATTAATGTATCTTTATGTCCCTGGCACATGAATAAACTTTG
CTGTGGTTTACTAATCTAAAAAAAAAAAAAAAAGGGCGGCCGCT

TABLE 12

Oligonucleotides

| | | | |
|---|---|---|---|
| | | | SEQ |
| | | Target | ID |
| Curagen # | Sequence | Site | NO |

| | | | |
|---|---|---|---|
| CG94235-01-TK-AS1 | CGGGAAGCTCCAGGACGAAG | 45 | 33 |
| CG94235-01-TK-AS2 | CGAAGTGAGCCAGGGTGCAG | 66 | 34 |
| CG94235-01-TK-AS3 | GCACGCACAGCGAGTAGCTG | 162 | 35 |
| CG94235-01-TK-AS4 | GCACTGGTCGACCAGGTCAA | 659 | 26 |
| CG94235-01-TK-AS5 | ACATCTAATCTAGTTAGACG | 1316 | 37 |

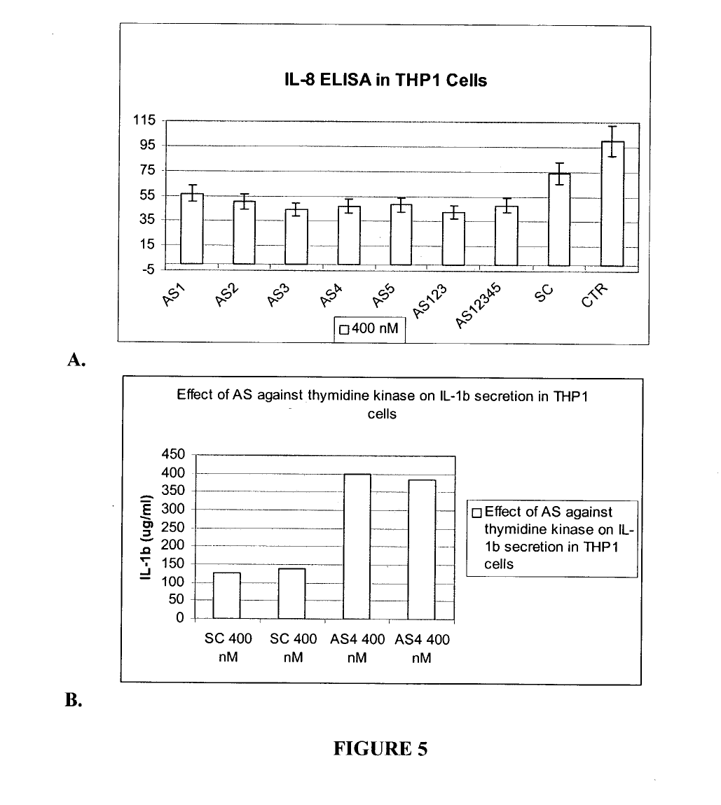

FIG. 5B is a bar graph showing changes in secretion of interleukin-1 (IL-β) protein via ELISA assay due to treatment with antisense (AS) nucleic acids specific for thymidine kinase compared to scatter control (SC) and control (CTR) nucleic acids in THP-1 cells.

### EXAMPLE 8

### Antisense Inhibition of H-ras mRNA Expression

A series of oligonucleotides were designed to target different regions of H-ras using the DNA sequence encoding a H-ras polypeptide The oligonucleotides are shown in Table 13. As described above, H-ras mRNA expression level in the absence or presence of M-B antisense oligonucleotides is shown in FIG. 2A. Suppression of T-24 cell proliferation at various concentrations of H-ras M-B antisense (RAS) compared to control is shown in FIG. 3A.

| | | |
|---|---|---|
| Curagen # | Sequence | SEQ ID NO |

| | | |
|---|---|---|
| H-RAS-AS1 | TCCGTCATCGCTCCTCAGGG | 38 |
| H-RAS-AS2 | CCCACCACCACCAGCTTATA | 39 |
| H-RAS-AS3 | TCAGCGCACTCTTGCCCACA | 40 |
| H-RAS-AS4 | CCACACCGACGGCGCCC | 41 |
| H-RAS-AS5 | TCAGGAGAGCACACACTTGC | 42 |

### EXAMPLE 9

### Antisense Inhibition of IL-8 Expression

A series of oligonucleotides were designed to target different regions of IL-8 using the DNA sequence encoding an IL-8 polypeptide. The oligonucleotides are shown in Table 14 As described above, IL-8 mRNA expression level in the absence or presence of M-B antisense oligonucleotides is shown in FIG. 2F.

| | | |
|---|---|---|
| Curagen # | Sequence | SEQ ID NO |

| | | |
|---|---|---|
| IL-8-AS1 | ACGGCCAGCTTGGAAGTCAT | 43 |
| IL-8-AS2 | GGAAGGCTGCCAAGAGAGCC | 44 |
| IL-8-AS3 | ACCTTCACACAGAGCTGCAG | 45 |
| IL-8-AS4 | CTCCACAACCCTCTGCACCC | 46 |
| IL-8-AS5 | CACTGGCATCTTCACTGATT | 47 |

FIG. 5A is a bar graph showing changes in secretion of interleukin-8 (IL-8) protein via ELISA assay due to treatment with antisense (AS) nucleic acids specific for IL-8 compared to scatter control (SC) and control (CTR) nucleic acids in THP-1 cells.

### EXAMPLE 10

### Western Immunoblot Analysis of Gene Suppression Due to Mixed Backbone Antisense DNA and Small Interfering RNA (siRNA).

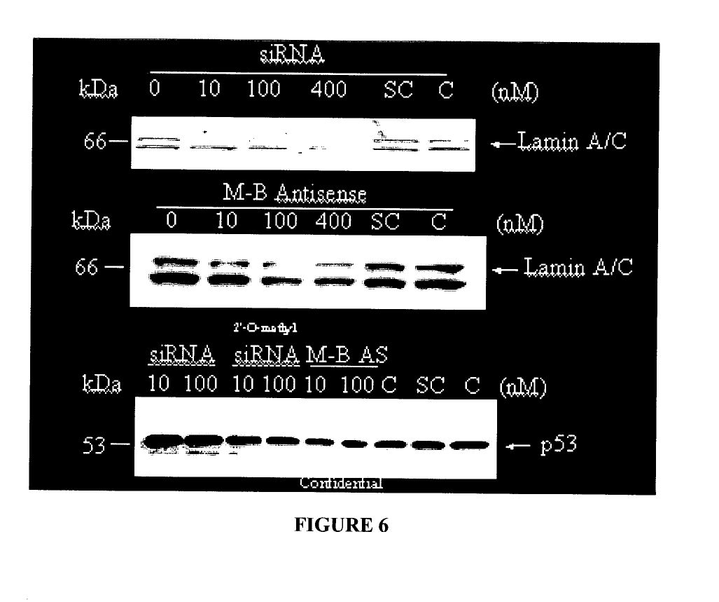

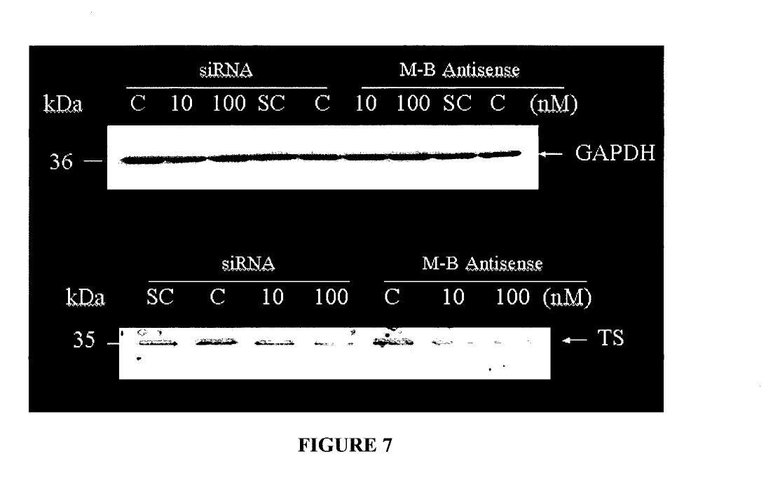

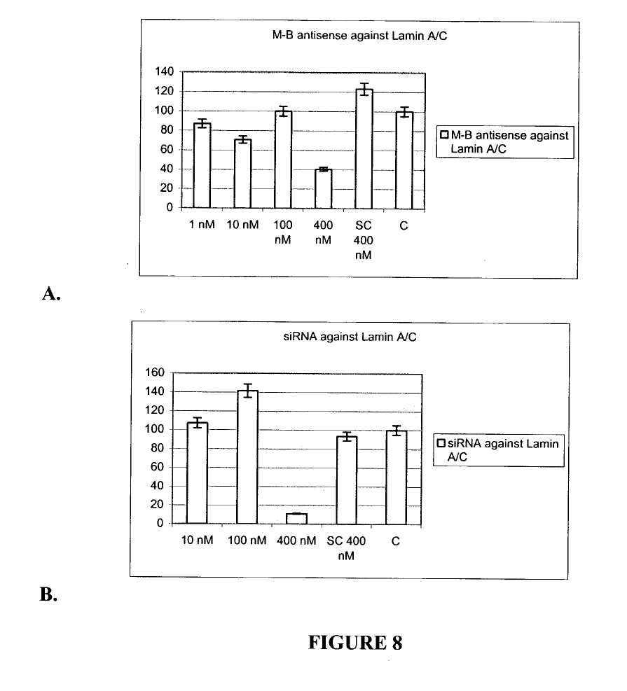

To compare the efficacy of M-B antisense oligo and siRNA for silencing gene expression, four genes were selected for gene knock out experiments. Both Hela-S3 and SW-620 cells were used to transfect M-B antisense oligos and siRNA using oligofectamine. Scramble control (SC) for both M-B antisense oligo and siRNA were used. Samples were then harvested for western immunoblot and TaqMan analysis for both protein and mRNA expression of the targeted genes. Instead of using 5 different M-B antisense oligos, single M-B antisense oligos were selected from the literature to target these 4 genes. Western immunoblot results are shown in FIGS. 6 and 7 and TaqMan results are shown in FIGS. 8-10.

FIG. 6 is a Western immunoblot of lamin A/C in Hela-S3 cells and p53 in SW-620 cells with increasing concentrations of mixed backbone (M-B) antisense DNA or small interfering RNA (siRNA). The siRNA marked O-methyl includes an O-methyl backbone. The M-B antisense oligo and siRNA successfully decreased Lamin A/C expression. The M-B antisense oligo had some effect on the expression of p53, but not siRNA.

FIG. 7 is a Western immunoblot of GAPDH and TS in SW-620 cells with varying concentrations of mixed backbone (M-B) antisense DNA or small interfering RNA (siRNA). M B antisense oligo and siRNA successfully decreased TS expression. Both M-B antisense oligos and siRNA have no effect on the expression of GAPDH.

FIG. 8 illustrates the change in lamin A/C mRNA with varying concentrations of antisense or interfering nucleic acids in HeLa-S3 cells measured via TaqMan. FIG. 8A shows changes in lamin A/C mRNA with varying concentrations of mixed backbone (M-B) antisense DNA, while FIG. 8B shows changes in lamin A/C mRNA with varying concentrations of small interfering RNA (siRNA).

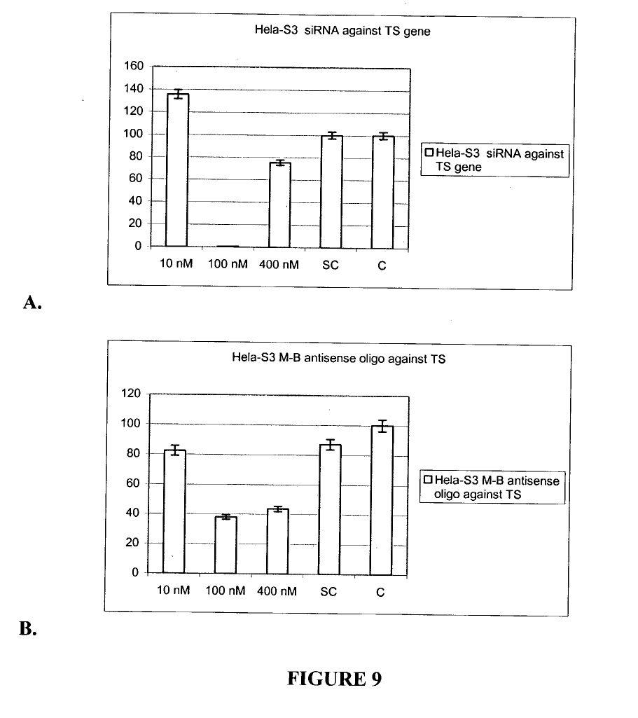

FIG. 9 illustrates the change in TS mRNA in response to varying concentrations of interfering or antisense nucleic acids in Hela-S3 cells. FIG. 9A shows changes in TS mRNA with varying concentrations of siRNA, while FIG. 9B graph shows changes in TS mRNA with varying concentrations of M-B antisense DNA.

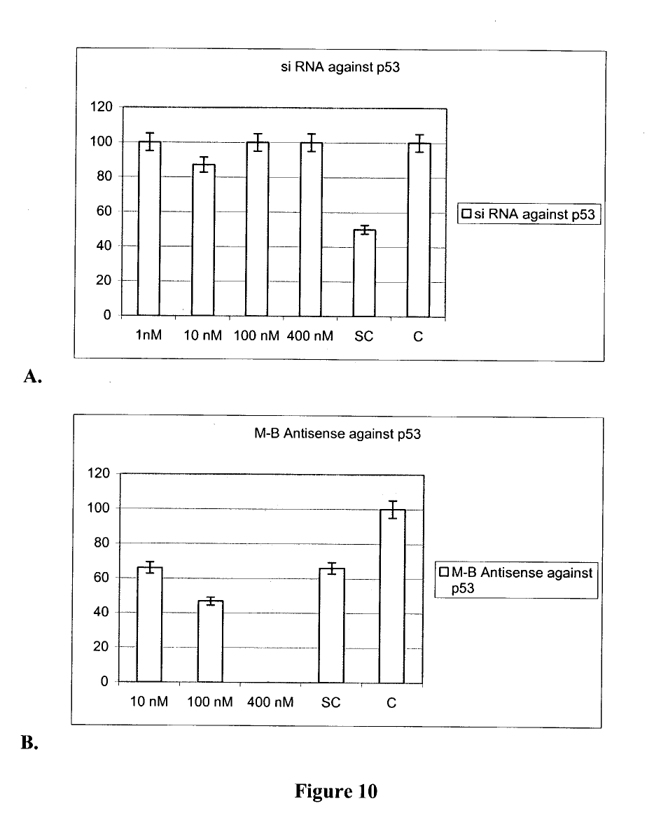

FIG. 10 illustrates the change in p53 mRNA with varying concentrations of interfering or antisense nucleic acids in cells. FIG. 10A shows changes in p53 mRNA with varying concentrations of siRNA, while FIG. 10B shows changes in p⁵³ mRNA with varying concentrations of M-B antisense DNA.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An oligonucleotide 20-50 nucleotides in length targeted to nucleotides 1-109 or nucleotides 434-513 of SEQ ID NO:1, wherein said oligonucleotide specifically hybridizes with one of said regions and inhibits expression of WNT-7B.

2. An oligonucleotide 10-50 nucleotides in length comprising at least 10 contiguous nucleotides of the nucleic acid sequence selected from the group consisting of SEQ ID NO: 7-11, wherein said oligonucleotide inhibits the expression of WNT-7B.

3. An oligonucleotide comprising the nucleic acid sequence of selected from the group consisting of SEQ ID NO: 7-11.

4. An oligonucleotide 20-50 nucleotides in length targeted to nucleotides 224-366, nucleotides 761-841, or 1062-1142 of SEQ ID NO:2, wherein said oligonucleotide specifically hybridizes with one of said regions and inhibits expression of N-acetylglucoaminyltransferase.

5. An oligonucleotide 10-50 nucleotides in length comprising at least 10 contiguous nucleotides of the nucleic acid sequence selected from the group consisting of SEQ ID NO: 12-16, wherein said oligonucleotide inhibits the expression of N-acetylglucoaminyltransferase.

6. An oligonucleotide comprising the nucleic acid sequence of selected from the group consisting of SEQ ID NO: 12-16.

7. An oligonucleotide 20-50 nucleotides in length targeted to nucleotides 1-121, nucleotides 1226-1201 or 1185-1953 of SEQ ID NO:3, wherein said oligonucleotide specifically hybridizes with one of said regions and inhibits expression of voltage gated K channel.

8. An oligonucleotide 10-50 nucleotides in length comprising at least 10 contiguous nucleotides of the nucleic acid sequence selected from the group consisting of SEQ ID NO: 17-21, wherein said oligonucleotide inhibits the expression of voltage gated K channel.

9. An oligonucleotide comprising the nucleic acid sequence of selected from the group consisting of SEQ ID NO: 17-21.

10. An oligonucleotide 20-50 nucleotides in length targeted to nucleotides 1-91, nucleotides 77-157, nucleotides 902-982 or nucleotides 1541-1621 of SEQ ID NO:4, wherein said oligonucleotide specifically hybridizes with one of said regions and inhibits expression of ion transport.

11. An oligonucleotide 10-50 nucleotides in length comprising at least 10 contiguous nucleotides of the nucleic acid sequence selected from the group consisting of SEQ ID NO: 23-27, wherein said oligonucleotide inhibits the expression of ion transport.

12. An oligonucleotide comprising the nucleic acid sequence of selected from the group consisting of SEQ ID NO: 23-27.

13. An oligonucleotide 20-50 nucleotides in length targeted to nucleotides 63-162 nucleotides 197-246, nucleotides 1037-1186 or nucleotides 1447-1526 of SEQ ID NO:5, wherein said oligonucleotide specifically hybridizes with one of said regions and inhibits expression of Map3K8.

14. An oligonucleotide 10-50 nucleotides in length comprising at least 10 contiguous nucleotides of the nucleic acid sequence selected from the group consisting of SEQ ID NO: 28-32, wherein said oligonucleotide inhibits the expression of Map3K8.

15. An oligonucleotide comprising the nucleic acid sequence of selected from the group consisting of SEQ ID NO: 28-32.

16. An oligonucleotide 20-50 nucleotides in length targeted to nucleotides 15-116 nucleotides 132-211, nucleotides 629-708 or nucleotides 1286-1165 of SEQ ID NO:6, wherein said oligonucleotide specifically hybridizes with one of said regions and inhibits expression of thymidine kinase.

17. An oligonucleotide 10-50 nucleotides in length comprising at least 10 contiguous nucleotides of the nucleic acid sequence selected from the group consisting of SEQ ID NO: 33-37, wherein said oligonucleotide inhibits the expression of thymidine kinase.

18. An oligonucleotide comprising the nucleic acid sequence of selected from the group consisting of SEQ ID NO: 33-37.

19. The oligonucleotide of claim 1, wherein said oligonucleotide comprises at least one modified internucleoside linkage.

20. The oligonucleotide of claim 1, wherein said oligonucleotide comprises at least one modified sugar moiety.

21. The oligonucleotide of claim 1, wherein said oligonucleotide comprises at least one modified nucleotide.

22. The oligonucleotide of claim 4, wherein said oligonucleotide comprises at least one modified internucleoside linkage.

23. The oligonucleotide of claim 4, wherein said oligonucleotide comprises at least one modified sugar moiety.

24. The oligonucleotide of claim 4, wherein said oligonucleotide comprises at least one modified nucleotide.

25. The oligonucleotide of claim 7, wherein said oligonucleotide comprises at least one modified internucleoside linkage.

26. The oligonucleotide of claim 7, wherein said oligonucleotide comprises at least one modified sugar moiety.

27. The oligonucleotide of claim 7, wherein said oligonucleotide comprises at least one modified nucleotide.

28. The oligonucleotide of claim 10, wherein said oligonucleotide comprises at least one modified internucleoside linkage.

29. The oligonucleotide of claim 10, wherein said oligonucleotide comprises at least one modified sugar moiety.

30. The oligonucleotide of claim 10, wherein said oligonucleotide comprises at least one modified nucleotide.

31. The oligonucleotide of claim 13, wherein said oligonucleotide comprises at least one modified internucleoside linkage.

32. The oligonucleotide of claim 13, wherein said oligonucleotide comprises at least one modified sugar moiety.

33. The oligonucleotide of claim 13, wherein said oligonucleotide comprises at least one modified nucleotide.

34. The oligonucleotide of claim 16, wherein said oligonucleotide comprises at least one modified internucleoside linkage.

35. The oligonucleotide of claim 16, wherein said oligonucleotide comprises at least one modified sugar moiety.

36. The oligonucleotide of claim 16, wherein said oligonucleotide comprises at least one modified nucleotide.

37. A method of inhibiting the expression of WNT-7B in a cell, comprising contacting said cell with the oligonucleotide of claim 1.

38. A method of inhibiting the expression of WNT-7B in a cell, comprising contacting said cell with one or more of the oligonucleotides of claim 2.

39. A method of inhibiting the expression of N-acetylglucoaminyltransferase in a cell, comprising contacting said cell with the oligonucleotide of claim 4.

40. A method of inhibiting the expression of N-acetylglucoaminyltransferase in a cell, comprising contacting said cell with one or more of the oligonucleotides of claim 5.

41. A method of inhibiting the expression of voltage gated K channel in a cell, comprising contacting said cell with the oligonucleotide of claim 7.

42. A method of inhibiting the expression of voltage gated K channel in a cell, comprising contacting said cell with one or more of the oligonucleotides of claim 8.

43. A method of inhibiting the expression of ion transport in a cell, comprising contacting said cell with the oligonucleotide of claim 10.

44. A method of inhibiting the expression of ion transport in a cell, comprising contacting said cell with one or more of the oligonucleotides of claim 11.

45. A method of inhibiting the expression of Map3K8 in a cell, comprising contacting said cell with the oligonucleotide of claim 13.

46. A method of inhibiting the expression of Map3K8 in a cell, comprising contacting said cell with one or more of the oligonucleotides of claim 14.

47. A method of inhibiting the expression of thymidine kinase in a cell, comprising contacting said cell with the oligonucleotide of claim 16.

48. A method of inhibiting the expression of thymidine kinase in a cell, comprising contacting said cell with one or more of the oligonucleotides of claim 17.

49. A method of inhibiting cell proliferation, comprising contacting a cell with the oligonucleotide of claim 1

50. A method of inhibiting cell proliferation, comprising contacting a cell with one or more of the oligonucleotides of claim 2.

51. The method of claim 49, wherein said oligonucleotide is present at a concentration of between 50 mM and 400 mM.

52. The method of claim 49, wherein said oligonucleotide comprises the nucleotide sequence selected from the group consisting of SEQ ID NO:7-11.

53. A method of inhibiting cell proliferation, comprising contacting a cell with the oligonucleotide of claim 4.

53. A method of inhibiting cell proliferation, comprising contacting a cell with one or more of the oligonucleotides of claim 5.

54. The method of claim 53, wherein said oligonucleotide is present at a concentration of between SOmM and 400 mM

55. The method of claim 53, wherein said oligonucleotide comprises the nucleotide sequence selected from the group consisting of SEQ ID NO: 12-16.

56. A method of increasing the production of Il-1b in a cell comprising contacting a cell with the oligonucleotide of claim 16.

57. A method of increasing the production of Il-1b in a cell, comprising contacting a cell with one or more of the oligonucleotides of claim 17.

58. The method of claim 53, wherein said oligonucleotide is present at a concentration of at least 400 mM.
